(19) United States
(12) Patent Application Publication
(10) Pub. No.: US 2024/0177012 A1
(43) Pub. Date: May 30, 2024

(54) Molecular Docking-Enabled Modeling of DNA-Encoded Libraries

(71) Applicant: Insitro, Inc., South San Francisco, CA (US)

(72) Inventors: Mohammad Muneeb Sultan, San Francisco, CA (US); Benson Chen, Palo Alto, CA (US); Kirill Shmilovich, Whitefish Bay, WI (US); Theofanis Karaletsos, San Francisco, CA (US)

(21) Appl. No.: 18/521,461

(22) Filed: Nov. 28, 2023

Related U.S. Application Data
(60) Provisional application No. 63/428,644, filed on Nov. 29, 2022.

(51) Int. Cl. G06N 3/09

(52) U.S. Cl. CPC G06N 3/09

(57) ABSTRACT

Embodiments of the disclosure involve training machine learned models using DNA-encoded library experimental data outputs and for deploying the trained machine learned models for conducting a virtual compound screen, for performing a hit selection and analysis, or for predicting binding affinities between compounds and targets. Machine learned models are trained using one or more augmentations that selectively expand molecular representations of a training dataset. Furthermore, machine learned models are trained to account for confounding covariates, thereby improving the machine learned models' abilities to conduct a virtual screen, perform a hit selection, and to predict binding affinities.

### CROSS-REFERENCE TO RELATED APPLICATIONS

This application claims the benefit of and priority to U.S. Provisional Patent Application No. 63/428,644 filed Nov. 29, 2022, the entire disclosure of which is hereby incorporated by reference in its entirety for all purposes.

### BACKGROUND OF THE INVENTION

DNA encoded libraries (DELs) are DNA barcode-labeled pooled compound collections that are incubated with an immobilized protein target in a process referred to as panning. The mixture is then washed to remove non-binders, and the remaining bound compounds are eluted, amplified, and sequenced to identify putative binders. DELs provide a quantitative readout for numerous (e.g., up to billions of) compounds.

Computational models have been deployed to learn the latent binding affinities that are loosely correlated to the sequenced count data of DEL experiments; however, the signal in DEL data is often obfuscated by various sources of noise introduced in its complicated data-generation process. When machine learning models are trained on data derived from DEL experiments, the noise and biases often contribute towards the poor performance of these models. Recent advances in DEL models have focused on probabilistic formulations of count data, but existing approaches have thus far been limited to only utilizing molecule-level representations. Thus, there is a need for improved methodologies for handling DEL experimental outputs to build improved machine learning models.

### SUMMARY

Disclosed herein are methods, non-transitory computer readable media, and systems for training machine learned models using DEL experimental datasets and for deploying the trained machine learned models for modeling binding affinity measurements (e.g., binding affinities between compounds and targets). In particular, disclosed herein is a new paradigm for modeling DELs that combines ligand-based descriptors with 3-D spatial information from docked compound-target complexes. Here, 3-D spatial information of docked compound-target complexes enables machine learning models to learn over the actual binding modality rather than using only molecule-based information of the compound. By incorporating 3-D spatial information, trained machine learning models are capable of effectively denoising DEL count data to predict target enrichment scores that are better correlated with experimental binding affinity measurements. Furthermore, an added benefit is that by learning over a collection of docked poses, machine learning models, trained on DEL data, implicitly learn to perform improved docking pose selection without requiring external supervision from expensive-to-source protein crystal structures.

Altogether, machine learned models disclosed herein are useful for various applications including conducting virtual compound screens, performing hit selection and analyses, and identifying common binding motifs. Conducting a virtual compound screen enables identifying compounds from a library (e.g., virtual library) that are likely to bind to a target, such as a protein target. Performing a hit selection enables identification of compounds that likely exhibit a desired activity. For example, a hit can be a compound that binds to a target (e.g., a protein target) and therefore, exhibits a desired effect by binding to the target. Predicting binding affinity between compounds and targets can result in the identification of compounds that exhibit a desired binding affinity. For example, binding affinity values can be continuous values and therefore, can be indicative of different types of binders (e.g., strong binder or weak binder). This enables the identification and categorization of compounds that exhibit different binding affinities to targets. Identifying common binding motifs can be useful for understanding the mechanism between binders of a target. An understanding of binding motifs can be useful for developing additional new small molecule compounds e.g., during medicinal chemistry campaigns.

Disclosed herein is a method for performing molecular screening of one or more compounds for binding to a target, the method comprising: obtaining a representation of a compound; obtaining a plurality of predicted compound-target poses and determining features of the plurality of the predicted compound-target poses; combining the representation of the compound and the features of the plurality of the predicted compound-target poses to generate a plurality of representations of compound-target poses; and analyzing, using a machine learning model, at least the plurality of representations of the compound-target poses to generate a target enrichment prediction representing binding between the compound and the target, and at least the representation of the compound to generate an off-target prediction. In various embodiments, the machine learning model comprises: a first portion trained to predict the target enrichment prediction from representations of compound-target poses; and a second portion trained to generate an off-target prediction from the representation of the compound. In various embodiments, methods further comprise predicting a measure of binding between the compound and the target using the target enrichment prediction. In various embodiments, methods further comprise ranking the compound according to the target enrichment prediction.

In various embodiments, analyzing, using the machine learning model, at least the plurality of representations of the compound-target poses comprises: analyzing, using a first portion of the machine learning model, the plurality of representations of the compound-target poses to identify one or more candidate compound-target poses representing likely 3D configurations of the compound when bound to the target. In various embodiments, the first portion of the machine learning model comprises a self-attention layer comprising one or more learnable attention weights for analyzing at least the plurality of representations of the compound-target poses. In various embodiments, methods disclosed herein further comprise using the one or more learnable attention weights, ranking the one or more candidate compound-target poses. In various embodiments, the first portion of the machine learning model comprises a layer that pays equal attention to each of the plurality of representations of the compound-target poses. In various embodiments, the first portion of the machine learning model comprises a multilayer perceptron (MLP). In various embodiments, the MLP of the first portion of the machine learning model comprises parameters that are learned through supervised training techniques. In various embodiments, the second portion of the machine learning model comprises a multilayer perceptron (MLP) to generate an off-target prediction from representations of compounds. In various embodiments, the MLP of the second portion of the machine learning model comprises parameters that are learned through supervised training techniques.

In various embodiments, the off-target prediction arises from one or more covariates comprising any of non-specific binding via controls, off-target data, and noise. In various embodiments, off-targets data comprise one or more of binding to beads, binding to streptavidin of the beads, binding to biotin, binding to gels, binding to DEL container surfaces. In various embodiments, the noise comprises one or more of starting tag imbalance, experimental conditions, chemical reaction yields, side and truncated products, errors from the library synthesis, DNA affinity to target, sequencing depth, and sequencing noise.

In various embodiments, the first portion of the machine learning model and the second portion of the machine learning model are trained using one or more training compounds with corresponding DNA-encoded library (DEL) outputs. In various embodiments, the corresponding DNA-encoded library (DEL) outputs for a training compound comprises: control counts arising from a covariate determined through a first panning experiment; and target counts determined through a second panning experiment. In various embodiments, for one of the training compounds, the first portion of the machine learning model and the second portion of the machine learning model are trained by: generating, by the first portion, a target enrichment prediction from representations of training compound-target poses, the representations of training compound-target poses generated by combining a representation of the training compound and features of a plurality of predicted training compound-target poses; generating, by the second portion, an off-target prediction from a representation of the training compound; combining the target enrichment prediction and the off-target prediction to generate a predicted target counts; and determining, according to a loss function, a loss value based on the predicted target counts and the experimental target counts. In various embodiments, the loss value is further determined based on the off-target predictions and the experimental control counts. In various embodiments, the loss function is any one of a negative log-likelihood loss, binary cross entropy loss, focal loss, arc loss, cosface loss, cosine based loss, or loss function based on a BEDROC metric. In various embodiments, the loss value is determined according to probability density functions that model the experimental target counts and the experimental control counts. In various embodiments, the probability density functions are represented by any one of a Poisson distribution, Binomial distribution, Gamma distribution, Binomial-Poisson distribution, Gamma-Poisson distribution, or negative binomial distribution. In various embodiments, the Poisson distribution is a zero-inflated Poisson distribution. In various embodiments, the loss value is determined by calculating a root mean squared error (RMSE) value.

In various embodiments, obtaining a plurality of predicted compound-target poses and determining features of the plurality of the predicted compound-target poses comprises performing an in silico molecular docking analysis. In various embodiments, performing the in silico molecular docking analysis generates the plurality of predicted compound-target poses. In various embodiments, performing the in silico molecular docking analysis determines the features of the plurality of predicted compound-target poses. In various embodiments, performing the in silico molecular docking analysis comprises applying one or more convolutional neural networks. In various embodiments, the plurality of predicted compound-target poses comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 compound-target poses. In various embodiments, the plurality of predicted compound-target poses comprises 20 compound-target poses. In various embodiments, obtaining the representation of the compound comprises: obtaining a molecular fingerprint for the compound; and optionally further comprises generating the representation of the molecular fingerprint. In various embodiments, generating the representation of the molecular fingerprint comprises applying a multilayer perceptron to the molecular fingerprint. In various embodiments, the molecular fingerprint is a Morgan fingerprint. In various embodiments, the representation of the compound is a neural network embedding of the compound. In various embodiments, each representation of the plurality of representations of compound-target poses is a neural network embedding of the compound-target pose.

In various embodiments, the measure of binding is any one of a binding affinity, DEL counts, DEL reads, or DEL indices. In various embodiments, the molecular screen is a virtual molecular screen. In various embodiments, the compound is from a virtual library of compounds. In various embodiments, the target comprises a protein target. In various embodiments, the protein target is a human carbonic anhydrase IX (CAIX) protein target.

In various embodiments, methods disclosed herein further comprise identifying a common binding motif across a subset of the one or more compounds, wherein the compounds in the subset have predicted measures of binding above a threshold binding value. In various embodiments, the common binding motif comprises a benzenesulfonamide.

Additionally disclosed herein is a method for performing molecular screening of one or more compounds for binding to a target, the method comprising: obtaining a representation of the compound; obtaining a plurality of predicted compound-target poses and determining features of the plurality of the predicted compound-target poses; combining the representation of the compound and the features of the plurality of the predicted compound-target poses to generate a plurality of representations of compound-target poses; analyzing, using a machine learning model, at least the plurality of representations of the compound-target poses to generate a target enrichment prediction representing target binding between the compound and the target; and predicting a measure of binding between the compound and the target using the target enrichment prediction. In various embodiments, the machine learning model is trained to learn separate contributions arising from noise-based sources and from target binding using spatial information of representations of compound-target poses and molecular level descriptors of molecular representations. In various embodiments, methods disclosed herein further comprise ranking the compound according to the target enrichment prediction.

In various embodiments, analyzing, using the machine learning model, at least the plurality of representations of the compound-target poses further comprises: analyzing, using a first portion of the machine learning model, the plurality of representations of the compound-target poses to generate a target enrichment prediction representing target binding between the compound and the target; and analyzing, using a second portion of the machine learning model, the representation of the compound to generate an off-target prediction. In various embodiments, analyzing, using a first portion of the machine learning model, the plurality of representations of the compound-target poses to generate a target enrichment prediction comprises: analyzing, using the first portion of the machine learning model, the plurality of representations of the compound-target poses to identify one or more candidate compound-target poses representing likely 3D configurations of the compound when bound to the target. In various embodiments, the first portion of the machine learning model comprises a self-attention layer comprising one or more learnable attention weights for analyzing at least the plurality of representations of the compound-target poses. In various embodiments, methods disclosed herein further comprise using the one or more learnable attention weights, ranking the one or more candidate compound-target poses.

In various embodiments, the first portion of the machine learning model comprises a layer that pays equal attention to each of the plurality of representations of the compound-target poses. In various embodiments, the first portion of the machine learning model comprises a multilayer perceptron (MLP). In various embodiments, the MLP of the first portion of the machine learning model comprises parameters that are learned through supervised training techniques. In various embodiments, the second portion of the machine learning model comprises a multilayer perceptron (MLP) to generate an off-target prediction from representations of compounds. In various embodiments, the MLP of the second portion of the machine learning model comprises parameters that are learned through supervised training techniques. In various embodiments, the off-target prediction arises from one or more covariates comprising any of non-specific binding via controls, off-target data, and noise. In various embodiments, off-targets data comprise one or more of binding to beads, binding to streptavidin of the beads, binding to biotin, binding to gels, binding to DEL container surfaces. In various embodiments, the noise comprises one or more of starting tag imbalance, experimental conditions, chemical reaction yields, side and truncated products, errors from the library synthesis, DNA affinity to target, sequencing depth, and sequencing noise. In various embodiments, the first portion of the machine learning model and the second portion of the machine learning model are trained using one or more training compounds with corresponding DNA-encoded library (DEL) outputs. In various embodiments, the corresponding DNA-encoded library (DEL) outputs for a training compound comprises: control counts arising from a covariate determined through a first panning experiment; and target counts determined through a second panning experiment.

In various embodiments, for one of the training compounds, the first portion of the machine learning model and the second portion of the machine learning model are trained by: generating, by the first portion, a target enrichment prediction from representations of training compound-target poses, the representations of training compound-target poses generated by combining a representation of the training compound and features of a plurality of predicted training compound-target poses; generating, by the second portion, an off-target prediction from a representation of the training compound; combining the target enrichment prediction and the off-target prediction to generate a predicted target counts; and determining, according to a loss function, a loss value based on the predicted target counts and the experimental target counts. In various embodiments, the loss value is further determined based on the off-target prediction and optionally the experimental control counts. In various embodiments, the loss function is any one of a negative log-likelihood loss, binary cross entropy loss, focal loss, arc loss, cosface loss, cosine based loss, or loss function based on a BEDROC metric. In various embodiments, the loss value is determined according to probability density functions that model the experimental target counts and the experimental control counts. In various embodiments, the probability density functions are represented by any one of a Poisson distribution, Binomial distribution, Gamma distribution, Binomial-Poisson distribution, Gamma-Poisson distribution, or negative binomial distribution. In various embodiments, the Poisson distribution is a zero-inflated Poisson distribution. In various embodiments, the loss value is determined by calculating a root mean squared error (RMSE) value.

In various embodiments, obtaining a plurality of predicted compound-target poses and determining features of the plurality of the predicted compound-target poses comprises performing an in silico molecular docking analysis. In various embodiments, performing the in silico molecular docking analysis generates the plurality of predicted compound-target poses. In various embodiments, performing the in silico molecular docking analysis determines the features of the plurality of predicted compound-target poses. In various embodiments, performing the in silico molecular docking analysis comprises applying one or more convolutional neural networks. In various embodiments, the plurality of predicted compound-target poses comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 compound-target poses. In various embodiments, the plurality of predicted compound-target poses comprises 20 compound-target poses. In various embodiments, obtaining the representation of the compound comprises: obtaining a molecular fingerprint for the compound; and optionally further comprises generating the representation of the molecular fingerprint.

In various embodiments, generating the representation of the molecular fingerprint comprises applying a multilayer perceptron to the molecular fingerprint. In various embodiments, the molecular fingerprint is a Morgan fingerprint. In various embodiments, the representation of the compound is a neural network embedding of the compound. In various embodiments, each representation of the plurality of representations of compound-target poses is a neural network embedding of the compound-target pose.

In various embodiments, the measure of binding is any one of a binding affinity, DEL counts, DEL reads, or DEL indices. In various embodiments, the molecular screen is a virtual molecular screen. In various embodiments, the compound is from a virtual library of compounds. In various embodiments, the target comprises a protein target. In various embodiments, the protein target is a human carbonic anhydrase IX (CAIX) protein target. In various embodiments, methods disclosed herein further comprise: identifying a common binding motif across a subset of the one or more compounds, wherein the compounds in the subset have predicted measures of binding above a threshold binding value. In various embodiments, the common binding motif comprises a benzenesulfonamide.

Additionally disclosed herein is a non-transitory computer readable medium comprising instructions that, when executed by a processor, cause the processor to: obtain a representation of a compound; obtain a plurality of predicted compound-target poses and determining features of the plurality of the predicted compound-target poses; combine the representation of the compound and the features of the plurality of the predicted compound-target poses to generate a plurality of representations of compound-target poses; and analyze, using a machine learning model, at least the plurality of representations of the compound-target poses to generate a target enrichment prediction representing binding between the compound and the target, and at least the representation of the compound to generate an off-target prediction. In various embodiments, a non-transitory computer readable medium disclosed herein comprises instructions that, when executed by a processor, cause the processor to perform methods disclosed herein.

Additionally disclosed herein is a system comprising: a processor; and a non-transitory computer readable medium comprising instructions that, when executed by the processor, cause the processor to: obtain a representation of a compound; obtain a plurality of predicted compound-target poses and determining features of the plurality of the predicted compound-target poses; combine the representation of the compound and the features of the plurality of the predicted compound-target poses to generate a plurality of representations of compound-target poses; and analyze, using a machine learning model, at least the plurality of representations of the compound-target poses to generate a target enrichment prediction representing binding between the compound and the target, and at least the representation of the compound to generate an off-target prediction. In various embodiments, a system comprises: a processor; and a non-transitory computer readable medium comprising instructions that, when executed by the processor, cause the processor to perform methods disclosed herein.

### BRIEF DESCRIPTION OF THE DRAWINGS

These and other features, aspects, and advantages of the present invention will become better understood with regard to the following description and accompanying drawings. It is noted that, wherever practicable, similar or like reference numbers may be used in the figures and may indicate similar or like functionality. For example, a letter after a reference numeral, such as “DEL experiment 115A,” indicates that the text refers specifically to the element having that particular reference numeral. A reference numeral in the text without a following letter, such as “DEL experiment 115,” refers to any or all of the elements in the figures bearing that reference numeral (e.g., “DEL experiment 115” in the text refers to reference numerals “DEL experiment 115A” and/or “DEL experiment 115B” in the figures).

FIG. 1A depicts an example system environment involving a compound-target analysis system, in accordance with an embodiment.

FIG. 1B depicts an example DNA-Encoded Library (DEL) panning experiment, in accordance with an embodiment.

FIG. 2 depicts a block diagram of a compound-target analysis system, in accordance with an embodiment.

FIG. 3A depicts a flow diagram for implementing a machine learning model to generate a target enrichment prediction, in accordance with an embodiment.

FIG. 3B depicts a flow diagram showing the implementation of a machine learning model including a first model portion and a second model portion, for generating a target enrichment prediction, in accordance with an embodiment.

FIG. 4 depicts an example flow process for implementing a machine learning model, in accordance with an embodiment.

FIG. 5 depicts an example flow diagram for training the machine learning model, in accordance with an embodiment.

FIG. 6 depicts an example flow process for training a machine learning model, in accordance with an embodiment.

FIG. 7A illustrates an example computing device for implementing the system and methods described in FIGS. 1A-1B, 2, 3A-3B, 4, 5, and 6

FIG. 7B depicts an overall system environment for implementing a compound-target analysis system, in accordance with an embodiment.

FIG. 7C is an example depiction of a distributed computing system environment for implementing the system environment of FIG. 7B.

FIG. 8A shows a comparison of distribution of molecular weights between the DEL data set and the full evaluation data set (left panel) and the 417-517 amu subset of the evaluation data set (right panel).

FIG. 8B shows a tSNE embedding of the DEL data set alongside the evaluation data.

FIG. 9 depicts a schematic illustration of the DEL-Dock neural network architecture and data flow.

FIG. 10A is a visual depiction showing that the model predicts sulfonamides within the evaluation data set as more highly enriched compared to molecules which do not contain benzenesulfonamides.

FIG. 10B shows a distribution of zinc-sulfonamide distances for the top-selected docked pose comparing AutoDock Vina, GNINA, and the DEL-dock method for all 1581 benzenesulfonamides-containing molecules in the evaluation data set.

FIG. 10C shows a cumulative distribution of the fraction of top-selected poses with zinc-sulfonamide distances below a distance threshold.

FIG. 11 shows an analysis of pose attention scores for a representative molecule in the evaluation data set.

FIG. 12 shows the distributions of zinc-sulfonamide distances throughout the top five ranking poses as identified by the DEL-DOCK model attention scores, GNINA pose selection, and the AutoDock Vina scoring function.

### DETAILED DESCRIPTION OF THE INVENTION

### Definitions

Terms used in the claims and specification are defined as set forth below unless otherwise specified.

The phrase “obtaining a representation of a compound” comprises generating a representation of a compound or obtaining a representation of the compound e.g., from a third party that generated the representation of the compound. Examples of a representation of the compound include a transformation of a molecular fingerprint or a molecular graph. An example transformation of a molecular fingerprint or a molecular graph can be a fingerprint embedding generated by applying a neural network. A compound may be in a particular structure format, including any of a simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format. Thus, a representation of the compound may be a transformation of the compound in a particular structure format.

The phrase “obtaining a plurality of predicted compound-target poses” comprises generating the plurality of predicted compound-target poses e.g., by predicting poses of the compound and target when they are bound. The phrase “obtaining a plurality of predicted compound-target poses” further comprises obtaining the plurality of predicted compound-target poses e.g., from a third party that generated the plurality of predicted compound-target poses.

The phrase “target enrichment prediction” refers to a prediction learnt by a machine learning model informative for a measure of binding between a compound and a target. In various embodiments, the target enrichment prediction is a value or a score. Generally, the target enrichment prediction is informative (e.g., correlated) for a measure of binding between a compound and a target, and a prediction that is denoised to account for an off-target prediction (e.g., absent influence from covariates and other sources of noise). In various embodiments, the target enrichment prediction is learned by attempting to predict the experimental DEL counts (which includes counts arising from sources of noise and covariates).

The phrase “off-target rediction” refers to a prediction learnt by a machine learning model that arises from non-target binding, such as the effects of one or more covariates and/or other sources of noise (e.g., sources of noise in DEL experiments). In various embodiments, the off-target prediction is a value or a score. Example covariates can include any of non-specific binding (e.g., as determined from controls) and other off-target data (e.g., binding to beads, binding to streptavidin of the beads, binding to biotin, binding to gels, binding to DEL container surfaces) or noise, such as starting tag imbalance, experimental conditions, chemical reaction yields, side and truncated products, errors from the library synthesis, DNA affinity to target, sequencing depth, and sequencing noise such as PCR bias.

It must be noted that, as used in the specification and the appended claims, the singular forms “a,” “an” and “the” include plural referents unless the context clearly dictates otherwise.

### Overview of System Environment

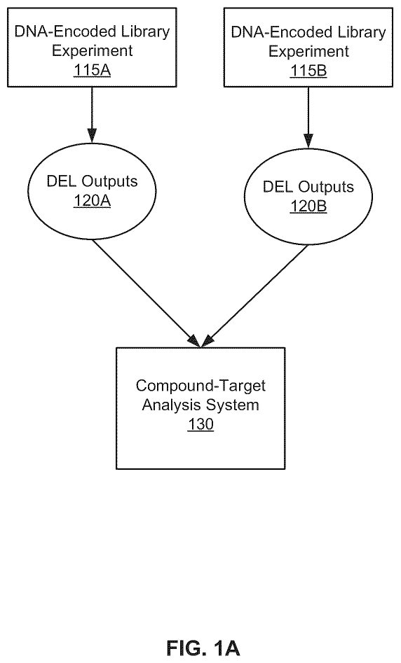

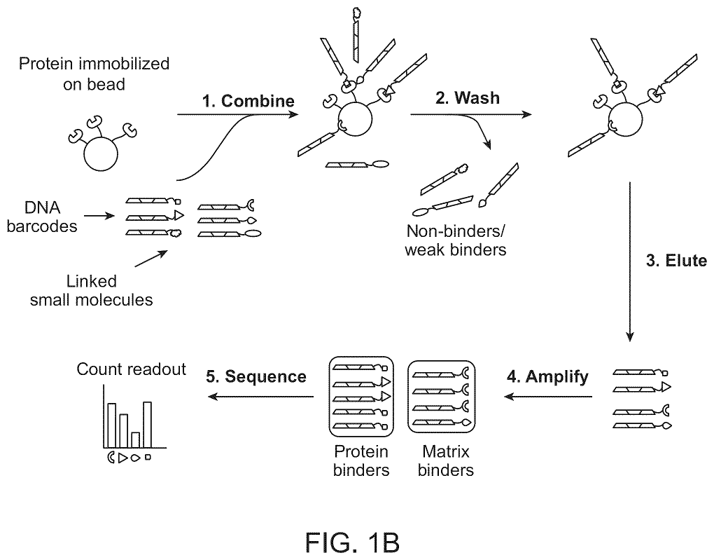

FIG. 1A depicts an example system environment involving a compound-target analysis system 130, in accordance with an embodiment. In particular, FIG. 1A introduces DNA-encoded library (DEL) experiment 115A and DNA-encoded library (DEL) experiment 115B for generating DEL outputs (e.g., DEL output 120A and DEL output 120B) that are provided to the compound-target analysis system 130 for training and deploying machine learning models. In particular embodiments, machine learning models are useful for generating target enrichment predictions which can be correlated to a measure of binding between compounds and targets e.g., for performing a virtual compound screen or for selecting and analyzing hits.

As shown in FIG. 1A, two DEL experiments 115A and 115B may be conducted. However, in various embodiments, fewer or additional DEL experiments can be conducted. In various embodiments, different DEL experiments 115A and 115B shown in FIG. 1A can refer to different replicates of similar/same experimental conditions. In various embodiments, the example system environment involves at least three DEL experiments, at least four DEL experiments, at least five DEL experiments, at least six DEL experiments, at least seven DEL experiments, at least eight DEL experiments, at least nine DEL experiments, at least ten DEL experiments, at least fifteen DEL experiments, at least twenty DEL experiments, at least thirty DEL experiments, at least forty DEL experiments, at least fifty DEL experiments, at least sixty DEL experiments, at least seventy DEL experiments, at least eighty DEL experiments, at least ninety DEL experiments, or at least a hundred DEL experiments. The output (e.g., DEL output) of one or more of the DEL experiments can be provided to the compound-target analysis system 130 for training and deploying machine learning models.

In various embodiments, a DEL experiment involves screening small molecule compounds of a DEL library against targets. In some embodiments, a DEL experiment involves screening multiple DEL libraries (e.g., in a single pool or across multiple pools). The DEL experiments (e.g., DEL experiments 115A or 115B) involve building small molecule compounds using chemical building blocks, also referred to as synthons. In various embodiments, small molecule compounds can be generated using two chemical building blocks, which are referred to di-synthons. In various embodiments, small molecule compounds can be generated using three chemical building blocks, which are referred to as tri-synthons. In various embodiments, small molecule compounds can be generated using four or more, five or more, six or more, seven or more, eight or more, nine or more, ten or more, fifteen or more, twenty or more, thirty or more, forty or more, or fifty or more chemical building blocks. In various embodiments, a DNA-encoded library (DEL) for a DEL experiment can include at least 10³ unique small molecule compounds. In various embodiments, a DNA-encoded library (DEL) for a DEL experiment can include at least 10⁴ unique small molecule compounds. In various embodiments, a DNA-encoded library (DEL) for a DEL experiment can include at least 10⁵ unique small molecule compounds. In various embodiments, a DNA-encoded library (DEL) for a DEL experiment can include at least 10⁶ unique small molecule compounds. In various embodiments, a DNA-encoded library (DEL) for a DEL experiment can include at least 10⁷ unique small molecule compounds. In various embodiments, a DNA-encoded library (DEL) for a DEL experiment can include at least 108 unique small molecule compounds. In various embodiments, a DNA-encoded library (DEL) for a DEL experiment can include at least 10⁹ unique small molecule compounds. In various embodiments, a DNA-encoded library (DEL) for a DEL experiment can include at least 10¹⁰ unique small molecule compounds. In various embodiments, a DNA-encoded library (DEL) for a DEL experiment can include at least 10¹¹ unique small molecule compounds. In various embodiments, a DNA-encoded library (DEL) for a DEL experiment can include at least 10¹² unique small molecule compounds.

Generally, the small molecule compounds in the DEL are labeled with tags. For example, the chemical building blocks of small molecule compounds (e.g., synthons) may be individually labeled with tags. Therefore, a small molecule compound may be labeled with multiple tags corresponding to the synthons that make up the small molecule compound. In various embodiments, the small molecule compound can be covalently linked to a unique tag. In various embodiments, the tags include nucleic acid sequences. In various embodiments, the tags include DNA nucleic acid sequences.

In various embodiments, for a DEL experiment (e.g., DEL experiment 115A or 115B), small molecule compounds that are labeled with tags are incubated with immobilized targets. In various embodiments, targets are nucleic acid targets, such as DNA targets or RNA targets. In various embodiments, targets are protein targets. In particular embodiments, protein targets are immobilized on beads. The mixture is washed to remove small molecule compounds that did not bind with the targets. The small molecule compounds that were bound to the targets are eluted and the corresponding tag sequences are amplified. In various embodiments, the tag sequences are amplified through one or more rounds of polymerase chain reaction (PCR) amplification. In various embodiments, the tag sequences are amplified using an isothermal amplification method, such as loop-mediated isothermal amplification (LAMP). The amplified sequences are sequenced to determine a quantitative readout for the number of putative small molecule compounds that were bound to the target. Further details of the methodology of building small molecule compounds of DNA-encoded libraries and methods for identifying putative binders of a DEL target are described in McCloskey, et al. “Machine Learning on DNA-Encoded Libraries: A New Paradigm for Hit Finding.” J. Med. Chem. 2020, 63, 16, 8857-8866, and Lim, K. et al “Machine learning on DNA-encoded library count data using an uncertainty-aware probabilistic loss function.” arXiv: 2108.12471, each of which is hereby incorporated by reference in its entirety.

Reference is made to FIG. 1B, which depicts an example DNA-Encoded Library (DEL) panning experiment, in accordance with an embodiment. DNA-encoded libraries (DELs) may be constructed by sequentially assembling molecular building blocks, aka synthons, into molecules tagged with unique DNA-barcode identifiers. These are shown in FIG. 1B as “linked small molecules” with DNA barcodes. Once synthesized, the library is tested for affinity against a target of interest (e.g., a protein target of interest) through a series of selection experiments. For example, as shown in FIG. 1B, the target of interest may be a protein immobilized on a bead.

An experiment, also referred to herein as panning, involves combining the DEL molecule into a solution of the immobilized target of interest (e.g., step 1 shown in FIG. 1B). Step 2 shown in FIG. 1B involves washing the resulting mixture for multiple rounds. Non-binders and weak binders are removed due to the wash. This procedure leaves members of the DEL that remain bound (e.g., bound to the target of interest or bound to other elements, such as the matrix). Step 3 involves eluting the DEL molecules that remain bound. The eluted DEL molecules then undergo amplification at step 4. Of note, there may be some DEL molecules that were bound to the matrix (shown in FIG. 1B as “Matrix binders”) and therefore, did not wash away during the step 2 wash. These matrix binders may represent covariates and/or noise and are not actually binders to the target of interest. In contrast, the actual DEL molecules that are bound to the target of interest (shown in FIG. 1B as “Protein binders”) are also obtained.

At step 5, presence of the DEL molecules is subsequently identified using next-generation DNA sequencing. The resulting data after bioinformatics processing can include reads of the DNA and the corresponding molecules. Thus, the relative abundance (e.g., number of DEL counts) of the identified members of the DEL is, in theory, a reasonable proxy for their binding affinities.

In various embodiments, for a DEL experiment (e.g., DEL experiment 115A or 115B), small molecule compounds are screened against targets using solid state media that house the targets. Here, in contrast to panning-based systems which used immobilized targets on beads, targets are incorporated into the solid state media. For example, this screen can involve running small molecule compounds of the DEL through a solid state medium such as a gel that incorporates the target using electrophoresis. The gel is then sliced to obtain tags that were used to label small molecule compounds. The presence of a tag suggests that the small molecule compound is a putative binder to the target that was incorporated in the gel. The tags are amplified (e.g., through PCR or an isothermal amplification process such as LAMP) and then sequenced. Further details for gel electrophoresis methodology for identifying putative binders is described in International Patent Application No. PCT/US2020/022662, entitled “Methods and Systems for Processing or Analyzing Oligonucleotide Encoded Molecules,” which was filed Mar. 13, 2020 and is hereby incorporated by reference in its entirety.

In various embodiments, one or more of the DNA-encoded library experiments 115 are performed to model one or more covariates (e.g., off-target covariates or off-target predictions). Generally, a covariate refers to an experimental influence that impacts a DEL output (e.g., DEL counts) of a DEL experiment, and therefore serves as a confounding factor in determining the actual binding between a small molecule compound and a target. Example covariates include, without limitation, non-target specific binding (e.g., binding to beads, binding to matrix, binding to streptavidin of the beads, binding to biotin, binding to gels, binding to DEL container surfaces, binding to tags e.g., DNA tags or protein tags), and other off-target noise sources, such as enrichment in other negative control pans, enrichment in other target pans as indication for promiscuity, compound synthesis yield, reaction type, starting tag imbalance, initial load populations, experimental conditions, chemical reaction yields, side and truncated products, errors from the library synthesis, DNA affinity to target, sequencing depth, and sequencing noise such as PCR bias.

To provide an example, a DEL experiment 115 may be designed to model the covariate of small molecule compound binding to beads. Here, if a small molecule compound binds to a bead instead of or in addition to the immobilized target on the bead, the subsequent washing and eluting step may result in the detection and identification of the small molecule compound as a putative binder, even though the small molecule compound does not bind specifically to the target. Thus, a DEL experiment 115 for modeling the covariate of non-specific binding to beads may involve incubating small molecule compounds with beads without the presence of immobilized targets on the bead. The mixture of the small molecule compound and the beads is washed to remove non-binding compounds that did not bind with the beads. The small molecule compounds bound to beads are eluted and the corresponding tag sequences are amplified (e.g., amplified through PCR or isothermal amplification such as LAMP). The amplified sequences are sequenced to determine a quantitative readout for the number of small molecule compounds that were bound to the bead. Thus, this quantitative readout can be a DEL output (e.g., DEL output 120) from a DEL experiment (e.g., DEL experiment 115) that is then provided to the compound-target analysis system 130.

As another example, a DEL experiment 115 may be designed to model the covariate of small molecule compound binding to streptavidin linkers on beads. Here, the streptavidin linker on a bead is used to attach the target (e.g., target protein) to a bead. If a small molecule compound binds to the streptavidin linker instead of or in addition to the immobilized target on the bead, the subsequent washing and eluting step may result in the detection and identification of the small molecule compound as a putative binder, even though the small molecule compound does not bind specifically to the target. Thus, a DEL experiment 115 for modeling the covariate of non-specific binding to beads may involve incubating small molecule compounds with streptavidin linkers on beads without the presence of immobilized targets on the bead. The mixture of the small molecule compound and the streptavidin linker on beads is washed to remove non-binding compounds. The small molecule compounds bound to streptavidin linker on beads are eluted and the corresponding tag sequences are amplified (e.g., amplified through PCR or isothermal amplification such as LAMP). The amplified sequences are sequenced to determine a quantitative readout for the number of small molecule compounds that were bound to the streptavidin linkers on beads. Thus, this quantitative readout can be a DEL output (e.g., DEL output 120) from a DEL experiment (e.g., DEL experiment 115) that is then provided to the compound-target analysis system 130.

As another example, a DEL experiment 115 may be designed to model the covariate of small molecule compound binding to a gel, which arises when implementing the nDexer methodology. Here, if a small molecule compound binds to the gel during electrophoresis instead of or in addition to the immobilized target on the bead, the subsequent washing and eluting step may result in the detection and identification of the small molecule compound as a putative binder, even though the small molecule compound does not bind to the target. Thus, the DEL experiment 115 may involve incubating small molecule compounds with control gels that do not incorporate the target. The small molecule compounds bound or immobilized within the gel are eluted and the corresponding tag sequences are amplified (e.g., amplified through PCR or isothermal amplification such as LAMP). The amplified sequences are sequenced to determine a quantitative readout for the number of small molecule compounds that were bound or immobilized in the gel. Thus, this quantitative readout can be a DEL output (e.g., DEL output 120) from a DEL experiment (e.g., DEL experiment 115) that is then provided to the compound-target analysis system 130.

In various embodiments, at least two of the DEL experiments 115 are performed to model at least two covariates. In various embodiments, at least three DEL experiments 115 are performed to model at least three covariates. In various embodiments, at least four DEL experiments 115 are performed to model at least four covariates. In various embodiments, at least five DEL experiments 115 are performed to model at least five covariates. In various embodiments, at least six DEL experiments 115 are performed to model at least six covariates. In various embodiments, at least seven DEL experiments 115 are performed to model at least seven covariates. In various embodiments, at least eight DEL experiments 115 are performed to model at least eight covariates. In various embodiments, at least nine DEL experiments 115 are performed to model at least nine covariates. In various embodiments, at least ten DEL experiments 115 are performed to model at least ten covariates. The DEL outputs from each of the DEL experiments can be provided to the compound-target analysis system 130. In various embodiments, the DEL experiments 115 for modeling covariates can be performed more than once. For example, technical replicates of the DEL experiments 115 for modeling covariates can be performed. In particular embodiments, at least three replicates of the DEL experiments 115 for modeling covariates can be performed.

The DEL outputs (e.g., DEL output 120A and/or DEL output 120B) from each of the DEL experiments can include DEL readouts for the small molecule compounds of the DEL experiment. In various embodiments, a DEL output can be a DEL count for the small molecule compounds of the DEL experiment. Thus, small molecule compounds that are putative binders of a target would have higher DEL counts in comparison to small molecule compounds that are not putative binders of the target. As an example, a DEL count can be a unique molecular index (UMI) count determined through sequencing. As an example, a DEL count may be the number of counts observed in a particular index of a solid state media (e.g., a gel). In various embodiments, a DEL output can be DEL reads corresponding to the small molecule compounds. For example, a DEL read can be a sequence read derived from the tag that labeled a corresponding small molecule compound. In various embodiments, a DEL output can be a DEL index. For example, a DEL index can refer to a slice number of a solid state media (e.g., a gel) which indicates how far a DEL member traveled down the solid state media.

Generally, the compound-target analysis system 130 trains and/or deploys machine learning models that jointly consider a representation of the compound and spatial 3D compound-target docking information. Such machine learning models are trained to learn latent binding affinity of compounds for targets and one or more covariates (e.g., the matrix). This leads to improved predictions by the machine learning models in the form of higher enrichment scores, which are well-correlated with compound-target binding affinity. Thus, such machine learning models trained and/or deployed by the compound-target analysis system 130 are useful for predicting anticipated target binding in virtual compound screening campaigns.

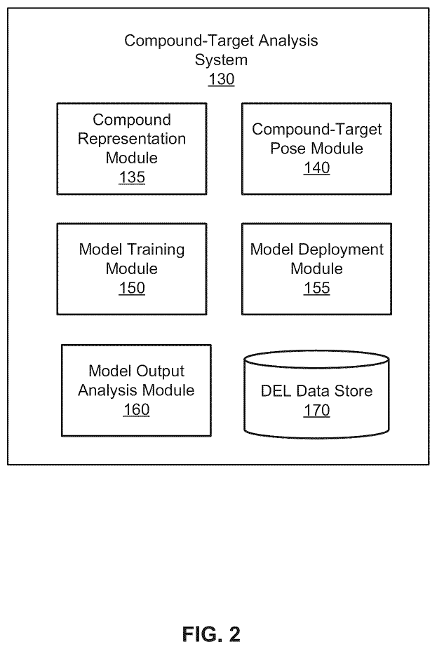

FIG. 2 depicts a block diagram of the compound-target analysis system 130, in accordance with an embodiment. FIG. 2 introduces individual components of the compound-target analysis system 130, examples of which include a compound representation module 135, a compound-target pose module 140, a model training module 150, a model deployment module 155, a model output analysis module 160, and a DEL data store 170.

Referring to the compound representation module 135, it generates representations of compounds (e.g., compounds and/or training compounds). In various embodiments, the compound representation module 135 generates a representation of a compound by obtaining an encoding of the compound, such as a molecular fingerprint or a molecular graph of the compound. An example molecular fingerprint of the compound is a Morgan fingerprint of the compound. Additional example encodings of the compound can be expressed in a particular structure, such as any of a simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format. In various embodiments, the compound representation module 135 generates a representation of a compound by transforming the encoding of the compound. In various embodiments, the compound representation module 135 applies a machine learning model, such as a neural network, to transform the encoding of the compound into a molecule embedding. Further details of the methods performed by the compound representation module 135 are described herein.

Referring to the compound-target pose module 140, it obtains compound-target poses, extracts features from the compound-target poses, and generates representations of the compound-target poses. Compound-target poses include 3-D spatial data of docked compound-target complexes. For example, compound-target poses can refer to 3D conformations of the compound and the target (e.g., protein target) when the compound and target are complexed together. In various embodiments, the compound-target pose module 140 obtains compound-target poses that are generated by performing an in silico molecular docking analysis. The compound-target pose module 140 further featurizes (e.g., extracts features) from the compound-target poses. Thus, these features represent information that characterize the 3D spatial interaction between the compound and target across the poses. The compound-target pose module 140 combines the features of the compound-target poses with the representation of the compound, previously generated by the compound representation module 135. Thus, by combining the features of the compound-target poses and the representation of the compound, the compound representation module 135 generates representations of compound-target poses, which jointly represent molecule-level descriptors of the compound and the spatial information of the docked compound-target complex. Thus, the compound-target pose module 140 can provide the representations of compound-target poses for training/deployment of machine learning models, as is described in further detail herein. Further details of the methods performed by the compound-target pose module 140 are described herein.

Referring to the model training module 150, it trains machine learning models using a training dataset. Generally, the model training module 150 trains machine learning models to effectively denoise DEL experimental data to generate target enrichment predictions representing binding between compounds and targets. In particular, the methods disclosed herein involve training machine learning models to generate target enrichment predictions that are better correlated with binding measurements in comparison to prior works. Further details of the training processes performed by the model training module 150 are described herein.

Referring to the model deployment module 155, it deploys machine learning models to generate target enrichment predictions representing binding between compounds and targets. The target enrichment predictions are useful for various applications, such as for performing a virtual compound screen, for selecting and analyzing hits, and for identifying common binding motifs on targets (e.g., protein targets). Further details of the processes performed by the model deployment module 155 are described herein.

Referring to the model output analysis module 160, it analyzes the outputs of one or more trained machine learned models. In various embodiments, the model output analysis module 160 translates predictions outputted by a machine learned model to a value representing a measure of binding between a compound and a target. As a specific example, the model output analysis module 160 may translate a target enrichment prediction outputted by a machine learning model to a binding affinity value. In various embodiments, the model output analysis module 160 ranks compounds according to their target enrichment predictions or according to the measure of binding. In various embodiments, the model output analysis module 160 identifies candidate compounds that are likely binders of a target based on the target enrichment prediction outputted by a machine learned model. For example, candidate compounds may be highly ranked compounds according to their target enrichment predictions or according to their measure of binding. Thus, candidate compounds can be synthesized e.g., as part of a medicinal chemistry campaign, and experimentally screened against the target to validate its binding and effects. Further details of the processes performed by the model output analysis module 160 are described herein.

### Example Methods for Generating Target Enrichment Predictions

As described herein, methods for generating target enrichment predictions involve training and/or deploying machine learning models that jointly analyze information of molecular-level descriptors and information of 3D spatial conformations of compounds and targets. Machine learning models are able to generate target enrichment predictions that better correlate with experimental binding affinity measurements.

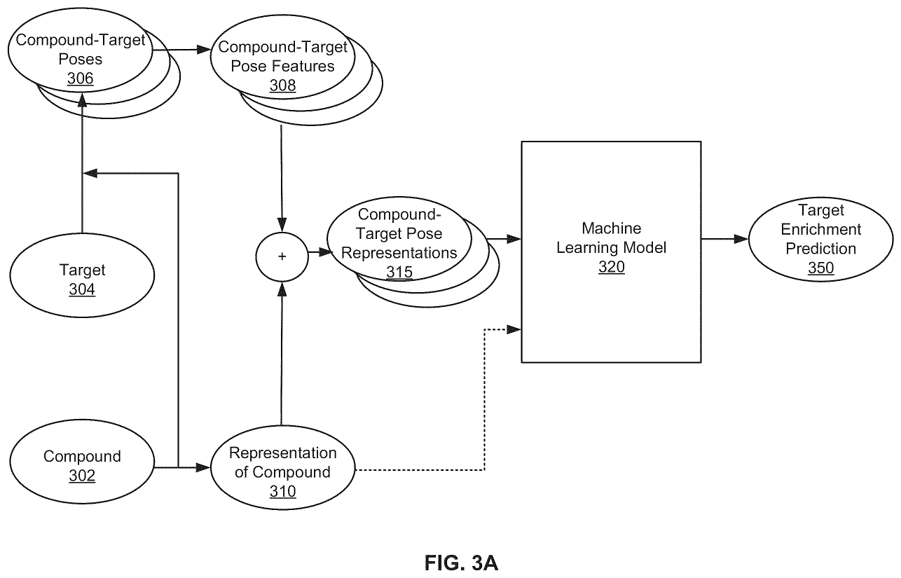

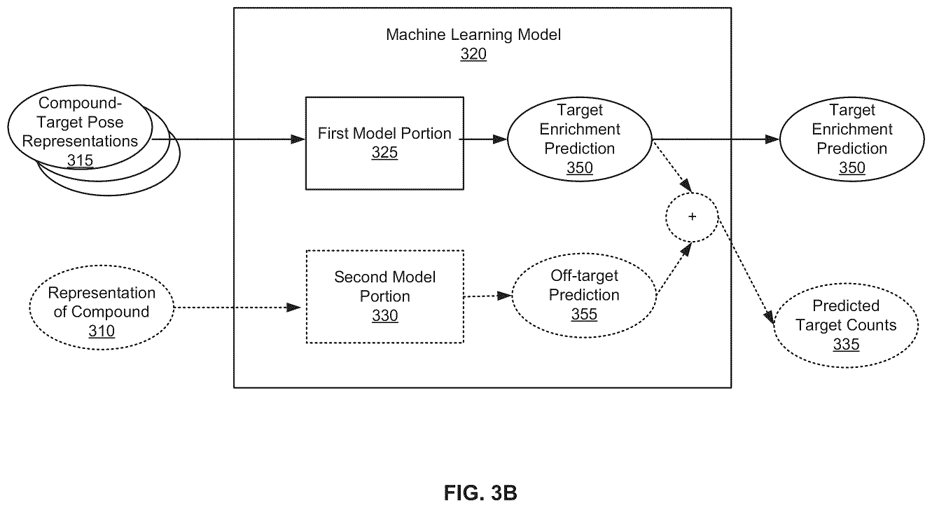

Reference is now made to FIG. 3A, which depicts a flow diagram for implementing a machine learning model to generate a target enrichment prediction, in accordance with an embodiment. FIG. 3A begins with a compound 302 and a target 304 (e.g., protein target). For example, the compound 302 may be included as a part of a virtual library of compounds for performing a molecular screen (e.g., a virtual molecular screen) against the target 304. In various embodiments, the target 304 can be a protein target. In particular embodiments, the target 304 can be a human protein target. The protein target may be implicated in disease and therefore, the virtual molecular screen is useful for identifying candidate compounds that can bind to the protein target and modulate its behavior in disease. As one specific example, the protein target may be a human carbonic anhydrase IX (CAIX) protein target. However, as one of skill in the art would appreciate, other known target proteins can be used.

In various embodiments, the compound 302 is an encoding of the compound, such as a molecular fingerprint or a molecular graph of the compound. In particular embodiments, the compound 302 is a Morgan fingerprint of the compound. In particular embodiments, let Φ: X→[0,1]ⁿ^{_{ϕ}} define the function that generates a n_{ϕ}-bit molecular fingerprint, where X denotes the set of compounds, where each compound x∈X. An example molecular fingerprint can be a 2048-bit Morgan fingerprint.

As shown in FIG. 3A, the compound 302 undergoes a transformation (e.g., performed by the compound representation module 135 as described in FIG. 2) to generate a representation of the compound 310. In particular embodiments, the compound representation module 135 applies a feedforward artificial neural network (ANN), an example of which is a multilayer perceptron (MLP), to transform the encoding of the compound to generate the representation of the compound 310. In such embodiments, the representation of the compound may be a neural network embedding of the compound. For example, let h^{f}=MLP(Φ(x)) be the molecule embedding (e.g., representation of the compound 310), which is computed by applying a multilayer perceptron (MLP) to the Morgan fingerprint. In various embodiments, the compound representation module 135 transforms the compound 302 to generate more than one representation of the compound 310. For example, the compound representation module 135 transforms the compound 302 to generate two or more, three or more, four or more, five or more, six or more, seven or more, eight or more, nine or more, or ten or more representations of the compound.

Returning to FIG. 3A, the compound 302 and target 304 are combined to generate compound-target poses 306. Here, this step may be performed by the compound-target pose module 140 described above in reference to FIG. 2. The compound-target pose module 140 generates compound-target poses 306 by performing an in silico molecular docking analysis. Generally, an molecular docking analysis refers to a method for predicting preferred orientations of the compound and the target when complexed together. Example molecular docking include AutoDock, which is described in Trott, O., et al., AutoDock Vina: improving the speed and accuracy of docking with a new scoring function, efficient optimization, and multithreading. Journal of computational chemistry 2010, 31, 455-461, which is hereby incorporated by reference in its entirety, as well as GNINA, which is described in McNutt, A. T., et al., GNINA 1.0: molecular docking with deep learning. Journal of cheminformatics 2021, 13, 1-20, which is also hereby incorporated by reference in its entirety.

Let each molecule x∈X have an associated set of n docked poses {p₁, p₂, . . . , pₙ}∈P. In various embodiments, the compound-target pose module 140 obtains at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 compound-target poses for a compound-target pair. In particular embodiments, compound-target pose module 140 obtains 20 compound-target poses for a compound-target pair.

As shown in FIG. 3A, the compound-target poses 306 are featurized to generate compound-target pose features 308. In various embodiments, the compound-target pose module 140 extracts features of compound-target poses by applying a machine learning model, such as a neural network (e.g., a convolutional neural network). In particular embodiments, the compound-target pose module 140 applies a pretrained GNINA convolutional neural network to extract features of compound-target poses. For example, let Ψ: X×P→Rⁿ^{_{Ψ}} define the transformation that outputs an embedding of the compound and a specific spatial protein-ligand complex, where a pre-trained voxel-based CNN (e.g., GNINA CNN) is used to perform this transformation. In some embodiments, the compound-target pose module 140 applies an untrained neural network (e.g., in contrast to a pretrained GNINA convolutional neural network). In such embodiments, such an untrained neural network can be trained to recognize valuable features from the compound-target poses 306. In various embodiments, the neural network can be trained along with the machine learning model 320 through end to end training techniques.

As shown in FIG. 3A, the representation of the compound 310 and the compound-target pose features 308 are combined to generate compound-target pose representations 315. Thus, the compound-target pose representations 315 jointly represent molecule-level descriptors of the compound 302 and the 3D spatial information of the compound-target poses 306. In various embodiments, the representation of the compound 310 and the compound-target pose features 308 are combined by applying a machine learning model, such as a neural network. In particular embodiments, the representation of the compound 310 and the compound-target pose features 308 are combined by applying a feedforward artificial neural network (ANN), an example of which is a multilayer perceptron (MLP). In various embodiments, a compound-target pose representation 315 is a neural network embedding of the compound-target pose. As a specific example, an individual docked pose embedding hₚⁱ is computed while incorporating the molecule embedding h^{f} (e.g., representation of the compound 310) and is defined as: hₚⁱ=MLP([Ψ(x, pᵢ); h^{f}]).

As further shown in FIG. 3A, the compound-target pose representations 315 are provided as input to the machine learning model 320. Here, given that the compound-target pose representations 315 jointly represent molecule-level descriptors of the compound 302 and the 3D spatial information of the compound-target poses 306, the machine learning model considers both molecular-level information and 3D spatial information to generate the target enrichment prediction 350.

In various embodiments, the representation of compound 310 may be provided as input to the machine learning model 320. However, in some embodiments, the representation of compound 310 is optional and need not be provided as input to the machine learning model 320. In particular embodiments, the representation of compound 310 (e.g., a representation of a training compound) is provided to the machine learning model 320 only during training of the machine learning model 320, as denoted by the dotted lines shown in FIG. 3A. During deployment of the machine learning model 320, the representation of compound 310 need not be provided as input to the machine learning model 320.

Reference is now made to FIG. 3B, which depicts a flow diagram showing the implementation of a machine learning model including a first model portion and a second model portion, in accordance with an embodiment. In particular, FIG. 3B introduces a first model portion 325 that analyzes the compound-target pose representations 315 to generate a target enrichment prediction 350 and a second model portion 330 that analyzes the representation of the compound 310 to generate an off-target prediction (e.g., a noise prediction) 355. The target enrichment prediction 350 and the off-target prediction 355 are combined to generate predicted target counts 335.

In various embodiments, all of the steps shown in FIG. 3B pertaining to the machine learning model 320 may be performed during training of the machine learning model 320 and during deployment of the machine learning model 320. However, in some embodiments, as indicated in FIG. 3B by the dotted lines, certain steps shown in FIG. 3B may only be performed when training the machine learning model 320 and need not be performed during deployment of the machine learning model 320. For example, the second model portion 330 may only be implemented during training of the machine learning model 320. Thus, during deployment, the second model portion 330 need not analyze the representation of the compound 310. In this scenario, neither the off-target prediction 355 nor the predicted target counts 335 are generated. In various embodiments, during deployment of the machine learning model 320, the off-target prediction 355 is generated, but is discarded and need not be used. In such embodiments, the predicted target counts 335 are not generated.

Referring to the first model portion 325, it analyzes the compound-target pose representations 315 and generates a target enrichment prediction 350. In various embodiments, the first model portion 325 involves one or more layers of a neural network. In various embodiments, the first model portion 325 involves one or more layers of a feedforward artificial neural network (ANN). In particular embodiments, the first model portion 325 includes one or more layers of a multilayer perceptron (MLP). In various embodiments, the first model portion 325 includes one or more layers of a transformer neural network. Such a transformer neural network exhibits an attention mechanism that enables the first model portion 325 to differently consider different subsets of the compound-target pose representations 315. For example, the attention mechanism enables the first model portion 325 to focus on certain subsets of the compound-target pose representations 315 over other subsets to generate the target enrichment prediction 350. In particular embodiments, the first model portion 325 includes one or more layers of a multilayer perceptron (MLP) and one or more layers of a transformer neural network. For example, the first model portion 325 includes layers of a MLP followed by layers of a transformer neural network. In such embodiments, the layers of the MLP transform the compound-target pose representations 315 into an intermediate representation that is then analyzed by the layers of the transformer neural network.

In various embodiments, the attention mechanism of a transformer neural network of the first model portion 325 involves an attention weight (e.g., learnable weight). In various embodiments, the attention mechanism of the first model portion 325 involves two, three, four, five, six, seven, eight, nine, or ten attention weights (e.g., learnable weights). In particular embodiments, the attention mechanism of the first model portion 325 involves three attention weights (e.g., learnable weights). Thus, the first model portion 325 combines the attention weights with the compound-target pose representations 315 to generate the target enrichment prediction 350. In various embodiments, the target enrichment prediction 350 is an attention-score weighted embedding vector. As one example, three attention weights of the first model portion 325, denoted as (w^{T}, W^{U}, and W^{V}), are computed according to Equation (1) described below in the Examples.

In various embodiments, the first model portion 325 analyzes the compound-target pose representation 315 and identifies one or more candidate compound-target poses that represent likely 3D configurations of the compound when bound to the target. Here, the first model portion 325 can identify the one or more candidate compound-target poses using attention weights (e.g., attention weights of the transformer neural network). For example, as the first model portion 325 performs self-attention over the compound-target pose representations 315, the magnitude of the attention weights can be interpreted as the importance of particular compound-target poses. Thus, compound-target poses associated with higher attention weights can be identified as candidate compound-target poses. In various embodiments, methods involve ranking the one or more candidate compound-target poses according to their attention weights (e.g., candidate compound-target poses associated with higher attention weights are more highly ranked in comparison to candidate compound-target poses associated with lower attention weights). Of note, the benefit of this approach is that the first model portion 325 can learn to identify likely compound-target poses in an unsupervised manner, without requiring scarce and expensive crystal structures to serve as the source of supervision for pose selection.

Generally, the target enrichment prediction 350 is a prediction that jointly utilizes information of the compound and 3D spatial compound-target docking information. For example, the target enrichment prediction 350 is learned through λₜ=f(h^{f}, h^{p}), where h^{f} denotes the representation of the compound (e.g., molecule embedding) and h^{p} denotes the attention-score weighted embedding vector computed from Equation (1) described below in the Examples. This design choice reflects that sequencing counts from the target protein DEL experiment is a function of the compound, as well as compound binding to the target, represented as embeddings derived from featurizations of the docked protein-ligand complexes. Specifically, the target enrichment prediction 350 is a prediction learnt by the machine learning model 320, the target enrichment prediction 350 representing a measure of binding between a compound and a target. For example, the target enrichment prediction represents a prediction of binding between a compound and a target that is denoised (e.g., absent influence from covariates and other sources of noise).

Referring to the second model portion 330, it analyzes the representation of the compound 310 and generates an off-target prediction (e.g., a noise prediction) 355. In various embodiments, the second model portion 330 includes one or more layers of a neural network. For example, the second model portion 330 includes one or more layers of a feedforward artificial neural network (ANN). In particular embodiments, the second model portion 330 includes one or more layers of a multilayer perceptron (MLP). As described herein, the off-target prediction 355 refers to a learnt prediction of the effects of one or more covariates (e.g., sources of noise in DEL experiments). For example, the off-target prediction can be a learnt prediction of the effects from one or more covariates comprising any of non-specific binding (e.g., as determined from controls) and/or other target data (e.g., binding to beads, binding to streptavidin of the beads, binding to biotin, binding to gels, binding to DEL container surfaces) or other sources of noise, such as, starting tag imbalance, experimental conditions, chemical reaction yields, side and truncated products, errors from the library synthesis, DNA affinity to target, sequencing depth, and sequencing noise such as PCR bias. Here, since DEL outputs arising from covariates are not a function of the compound-target pose, the off-target prediction (e.g., a noise prediction) 355 is computed by the second model portion 330 solely as a function of the representation of the compound 310 (e.g., molecule embedding h^{f}).

As shown in FIG. 3B, the target enrichment prediction 350 generated by the first model portion 325 may, in various embodiments, be directly outputted by the machine learning model 320. Thus, the target enrichment prediction 350 may be used to calculate a binding affinity value for the compound-target complex, as is discussed in further detail herein. Additionally or alternatively, the machine learning model 320 may calculate and output a predicted target counts 335. Here, the predicted target counts 335 may be a predicted DEL output for a DEL panning experiment. For example, the predicted target counts 335 may represent a DEL output of one or more DEL panning experiments, examples of which include a prediction of DEL counts and/or mean counts across multiple replicates of DEL panning experiments. Here, the predicted target counts 335 is a prediction of DEL counts in which various sources of off-target binding or noise (e.g., background, matrix, covariates) are included.

Generally, the target enrichment prediction 350 represents a measure of binding between the compound and the target and can be correlated to binding affinity. In various embodiments, the target enrichment prediction 350 can be converted to a binding affinity value. In various embodiments, the binding affinity value is measured by an equilibrium dissociation constant (K_{d}). In various embodiments, a binding affinity value is measured by the negative log value of the equilibrium dissociation constant (pK). In various embodiments, a binding affinity value is measured by an equilibrium inhibition constant (Kᵢ). In various embodiments, a binding affinity value is measured by the negative log value of the equilibrium inhibition constant (pKᵢ). In various embodiments, a binding affinity value is measured by the half maximal inhibitory concentration value (IC50). In various embodiments, a binding affinity value is measured by the half maximal effective concentration value (EC50). In various embodiments, a binding affinity value is measured by the equilibrium association constant (Kₐ). In various embodiments, a binding affinity value is measured by the negative log value of the equilibrium association constant (Kₐ). In various embodiments, a binding affinity value is measured by a percent activation value. In various embodiments, a binding affinity value is measured by a percent inhibition value.

In various embodiments, the target enrichment prediction 350 is converted to a binding affinity value according to a pre-determined conversion relationship. The pre-determined conversion relationship may be determined using DEL experimental data such as previously generated DEL outputs (e.g., DEL output 120A and 120B shown in FIG. 1A) based on DEL experiments. In various embodiments, the pre-determined conversion relationship is a linear equation. Here, the target enrichment prediction 350 may be correlated to the binding affinity value. In various embodiments, the pre-determined conversion relationship is any of a linear, exponential, logarithmic, non-linear, or polynomial equation.

In various embodiments, target enrichment predictions 350 can be used to rank order compounds. For example, a first compound with a target enrichment prediction that is correlated with a stronger binding affinity to a target can be ranked higher than a second compound with a target enrichment prediction that is correlated with a weaker binding affinity to the target. Generally, in a medicinal chemistry campaign such as hit-to-lead optimization, binding affinity values are commonly used to assess and select the next compounds to be synthesized. Thus, the target enrichment prediction, which correlates to binding affinity values, can be useful for rank ordering compounds and hence be used directly to guide design.

In various embodiments, the rank ordering of compounds is used to identify binders and non-binders. In various embodiments, identifying binders includes identifying the top Z compounds in the ranked list as binders. Compounds not included in the top Z compounds are considered non-binders. In various embodiments, the top Z compounds refers to any of the top 5 compounds, top 10 compounds, top 20 compounds, top 30 compounds, top 40 compounds, top 50 compounds, top 75 compounds, top 100 compounds, top 200 compounds, top 300 compounds, top 400 compounds, top 500 compounds, top 1000 compounds, or top 5000 compounds.

In various embodiments, compounds that are identified as binders to a target can be further analyzed to characterize the binders. In various embodiments, binders can be defined as compounds that have predicted binding affinity above a threshold binding value. In one scenario, binders are analyzed to identify common binding motifs in the binders that likely contribute towards effective binding between the binders and the target. In various embodiments, common binding motifs refer to chemical groups that appear in at least X % of the binders. In various embodiments, X % is at least 10% of binders, at least 20% of binders, at least 30% of binders, at least 40% of binders, at least 50% of binders, at least 60% of binders, at least 70% of binders, at least 80% of binders, at least 90% of binders, or at least 95% of binders. In various embodiments, X % is 100% of binders.

As a specific example, a target protein can be a human carbonic anhydrase IX (CAIX) protein. However, as one of skill in the art would appreciate, other known target proteins can be used. Using the methods described herein, compounds that bind to the target protein can be identified based on target enrichment predictions 350 generated by machine learning models. A binding motif that is commonly present in many of the compounds predicted to bind to the target protein (e.g., binders) can be a benzenesulfonamide group.

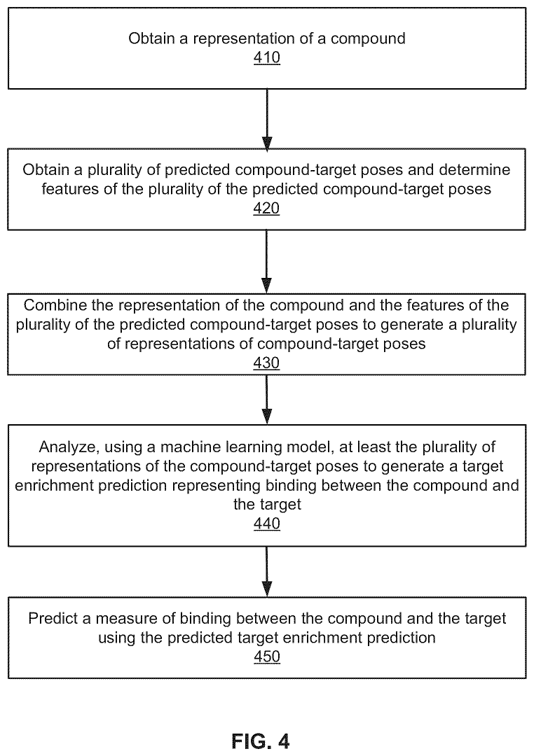

Reference is now made to FIG. 4, which depicts an example flow process for implementing a machine learning model, in accordance with an embodiment.

Step 410 involves obtaining a representation of a compound. For example, step 410 may involve obtaining a fingerprint, such as a Morgan fingerprint, of the compound. As another example, step 410 may involve obtaining a transformation of a fingerprint (e.g., a transformation of a Morgan fingerprint) of the compound. In various embodiments, the representation of the compound is a fingerprint embedding.

Step 420 involves obtaining a plurality of predicted compound-target poses and determining features of the plurality of the predicted compound-target poses. In particular embodiments, step 420 involves obtaining 20 or more compound-target poses, which represent possible 3D configurations of the compound when bound to the target. In various embodiments, determining features of the plurality of the predicted compound-target poses involves applying a neural network model that extracts features of the plurality of the predicted compound-target poses. In various embodiments, the neural network model is a pretrained model, such as a pretrained GNINA convolutional neural network. In some embodiments, the neural network model is not previously pre-trained and is, instead, trained along with machine learning models disclosed herein (e.g., machine learning model 320 shown in FIG. 3A) through end to end training.

Step 430 involves combining the representation of the compound and the features of the plurality of the predicted compound-target poses to generate a plurality of representations of compound-target poses. Thus, the plurality of representations of compound-target poses jointly represents both topological features of the compound as well as the spatial 3-D information of the compound-target complex.

Step 440 involves analyzing, using a machine learning model, at least the plurality of representations of the compound-target poses to generate a target enrichment prediction representing binding between the compound and the target. In various embodiments, analyzing at least the plurality of representations of the compound-target poses to generate a target enrichment prediction representing binding between the compound and the target comprises: analyzing, using a first portion of the machine learning model, the plurality of representations of the compound-target poses to identify one or more candidate compound-target poses representing likely 3D configurations of the compound when bound to the target; and analyzing, using the first portion of the machine learning model, the one or more candidate compound-target poses to generate the target enrichment prediction.

Step 450 involves predicting a measure of binding between the compound and the target using the predicted target enrichment prediction. In various embodiments, step 450 further involves ranking the compound according to the target enrichment prediction.

### Example Machine Learning Models

Embodiments disclosed herein involve training and/or deploying machine learning models for generating predictions for any of a virtual screen, hit selection and analysis, or predicting binding affinity. Generally, machine learning models disclosed herein jointly consider a representation of a compound and spatial 3D compound-target docking information to generate a prediction (e.g., a target enrichment prediction) that is correlated with binding affinity. In various embodiments, machine learning models disclosed herein can be any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, attention based models, geometric neural networks, equivariant neural networks, or recurrent networks (e.g., long short-term memory networks (LSTM), bi-directional recurrent networks, deep bi-directional recurrent networks).

In particular embodiments, machine learning models disclosed herein are neural networks, such as convolutional neural networks. A machine learning model may comprise different model portions e.g., a first portion, a second portion, and so on. In various embodiments, a machine learning model may include two portions. In various embodiments, a machine learning model may include three portions. In various embodiments, a machine learning model may include four portions, five portions, six portions, seven portions, eight portions, nine portions, or ten portions. Each portion of the machine learning model may have a different functionality. For example, as described herein, a first portion of the machine learning model may be trained to generate a target enrichment prediction from representations of compound-target poses. A second portion of the machine learning model may be trained to generate an off-target prediction from representations of compounds. Returning again to the context in which the machine learning model is a neural network, each portion of the machine learning model may be an individual set of layers. For example, a first portion of the machine learning model may refer to a first set of layers. A second portion of the machine learning model may refer to a second set of layers.

In various embodiments, the different portions of the machine learning model can be differently employed during training and deployment phases. For example, during training, both the first and second portions of the machine learning model are implemented to learn parameters that enable the machine learning model to generate target enrichment predictions. During deployment, the first portion of the machine learning model can be deployed to generate target enrichment predictions, but the second portion of the machine learning model need not be deployed.

In various embodiments, machine learning models disclosed herein can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient based optimization technique, and dimensionality reduction techniques such as manifold learning, principal component analysis, factor analysis, autoencoder, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, machine learning models disclosed herein have one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of a neural network, support vectors in a support vector machine, and coefficients in a regression model. The model parameters of the machine learning model are trained (e.g., adjusted) using the training data to improve the predictive power of the machine learning model.

### Training Machine Learning Models

Embodiments disclosed herein describe the training of machine learned models that jointly consider a representation of compounds and spatial 3D compound-target information. Generally, machine learning models are trained to generate target enrichment predictions, which represent the learnt binding strength between compounds and targets. Thus, the target enrichment prediction can be useful for identifying and/or ranking potential binders e.g., in virtual compound screens. In various embodiments, the target enrichment prediction represents an intermediate prediction of a machine learning model. For example, the target enrichment prediction is learned by training the machine learning model to predict the experimentally observed target counts and/or experimentally observed control counts arising from background/matrix/covariates.

As described herein, in various embodiments, the machine learning model includes at least a first portion and a second portion. The first portion of the machine learning model is trained to generate the target enrichment prediction and the second portion is trained to generate an off-target prediction (e.g., a noise prediction). Here, the first portion of the machine learning model may include a first set of tunable parameters and the second portion of the machine learning model may include a second set of tunable parameters. Thus, during training of the machine learning model, first set of tunable parameters and the second set of tunable parameters can be adjusted to improve the predictions generated by the machine learning model. In various embodiments, the first set and second set of tunable parameters are jointly adjusted.

Generally, the first portion of the machine learning model and the second portion of the machine learning model are trained using training compounds with corresponding DNA-encoded library (DEL) outputs. As used herein, training compounds refer to compounds with known corresponding experimental counts generated through one or more DEL panning experiments. Thus, these experimental counts can represent ground truth values for training the machine learning model.

In various embodiments, a training compound has a known corresponding experimental target count from a DEL panning experiment. The experimental target count may refer to signal in DEL data from a DEL experiment in which various sources of noise (e.g., background, matrix, covariates) are included. For example, the DEL experiment may include immobilizing protein targets on beads, exposing the protein targets to DEL compounds, washing the mixture to remove unbound compounds, and eluting, amplifying, and sequencing the tag sequences. Thus, the experimental target count obtained from this DEL experiment may include data arising from the various sources of noise.

In various embodiments, a training compound has one or more known corresponding experimental control counts from a DEL panning experiment. The experimental control counts may refer to signal in DEL data from a DEL experiment in which only one or more sources of noise (e.g., background, matrix, covariates) are included. For example, a DEL experiment may model a covariate (e.g., non-specific binding to beads). This involves incubating small molecule compounds with beads without the presence of immobilized targets on the bead. The mixture is washed to remove non-binders, followed by elution, sequence amplification, and sequencing. Thus, the experimental control counts obtained from this DEL experiment includes data arising from the sources of noise, but does not include data arising from actual binding of compounds and the target.

In various embodiments, a training compound has both 1) one or more known corresponding experimental control counts from one or more additional DEL panning experiments and 2) a known corresponding experimental target count from a DEL panning experiment. Specifically, the corresponding DNA-encoded library (DEL) outputs for a training compound comprises: 1) experimental control counts arising from a covariate determined through a first panning experiment; and 2) experimental target counts determined through a second panning experiment. In such embodiments, both the experimental control counts and the experimental target counts can be used as reference ground truth values for training the machine learning model. For example, a machine learning model is trained to generate a target enrichment prediction by attempting to predict the experimental control counts and the experimental target counts observed for training compounds.

Generally, during a training iteration involving a training compound, the methods for training the machine learning model involve obtaining a representation of the training compound, obtaining a plurality of predicted training compound-target poses and determining features of the plurality of the predicted training compound-target poses; and combining the representation of the training compound and the features of the plurality of the predicted training compound-target poses to generate a plurality of representations of training compound-target poses. Here, the step of obtaining a representation of the training compound may be performed in a similar or same manner as was described above in reference to a compound during deployment of the machine learning model (e.g., as described in reference to FIG. 3A). The step of obtaining a plurality of predicted training compound-target poses and determining features of the plurality of the predicted training compound-target poses may be performed in a similar or same manner as was described above in reference to a compound during deployment of the machine learning model (e.g., as described in reference to FIG. 3A). Additionally, the step of combining the representation of the training compound and the features of the plurality of the predicted training compound-target poses to generate a plurality of representations of training compound-target poses may be performed in a similar or same manner as was described above in reference to a compound during deployment of the machine learning model (e.g., as described in reference to FIG. 3A).

Furthermore, during a training iteration involving the training compound, the first portion of the machine learning model and the second portion of the machine learning model are trained by: generating, by the first portion, a target enrichment prediction from representations of training compound-target poses, the representations of training compound-target poses generated by combining a representation of the training compound and features of a plurality of predicted training compound-target poses; generating, by the second portion, an off-target prediction from a representation of the training compound. Here, these steps may be performed in a similar or same manner as was described above in reference to a compound during deployment of the machine learning model (e.g., as described in reference to FIG. 3B).

Furthermore, during a training iteration involving the training compound, the first portion of the machine learning model and the second portion of the machine learning model are trained by: combining the target enrichment prediction and the off-target prediction to generate a predicted target counts; and determining, according to a loss function, a loss value. The loss value can then be used (e.g., backpropagated) to tune the parameters of the first portion and second portion of the machine learning model.

In various embodiments, the loss value is calculated using the predicted target counts and the experimental target counts. For example, the closer the predicted target counts is to the experimental target counts, the smaller the loss value. In various embodiments, the loss value is calculated using the off-target prediction and the experimental control counts. For example, the closer the off-target prediction is to the experimental control counts, the smaller the loss value. In various embodiments, the loss value is calculated using each of the predicted target counts, the experimental target counts, the off-target prediction, and the experimental control counts. In such embodiments, the closer the predicted target counts are to the experimental target counts and the closer the off-target prediction is to the experimental control counts, the smaller the loss value. In various embodiments, the loss value is determined by calculating a root mean squared error (RMSE) value. For example, the RMSE value may be calculated as the square root of the summation of 1) a difference between predicted target counts and experimental target counts and 2) a difference between an off-target prediction (e.g., a noise prediction) and experimental control counts.

In various embodiments, the loss value is determined according to probability density functions that model the experimental target counts and the experimental control counts. In various embodiments, the loss value is determined according to a first probability density function that models the experimental target counts and a second probability density function that models the experimental control counts.

In various embodiments, the probability density functions are represented by any one of a Poisson distribution, Binomial distribution, Gamma distribution, Binomial-Poisson distribution, or Gamma-Poisson distribution. In particular embodiments, the probability density functions are represented by Poisson distributions. In various embodiments, the Poisson distributions are zero-inflated Poisson distributions. As a specific example, a zero-inflated Poisson distribution can have a probability density function (PDF) defined according to Equation (2) described in the Examples below. Example zero-inflated Poisson (ZIP) distributions are described according to Equations (3) and (4) (e.g., C^{m} and C^{t}) in the Examples below. In particular embodiments, Poisson distributions are characterized according to a rate parameter λ. Example rate parameters λₘ and λₜ of Poisson distributions are described according to Equations (3) and (4) in the Examples below.

In various embodiments, the loss function is any one of a negative log-likelihood loss, binary cross entropy loss, focal loss, arc loss, cosface loss, cosine based loss, or loss function based on a BEDROC metric. In particular embodiments, the loss function is a negative log-likelihood loss. An example negative log-likelihood loss function is exemplified as Equation (5) described in the Examples below.

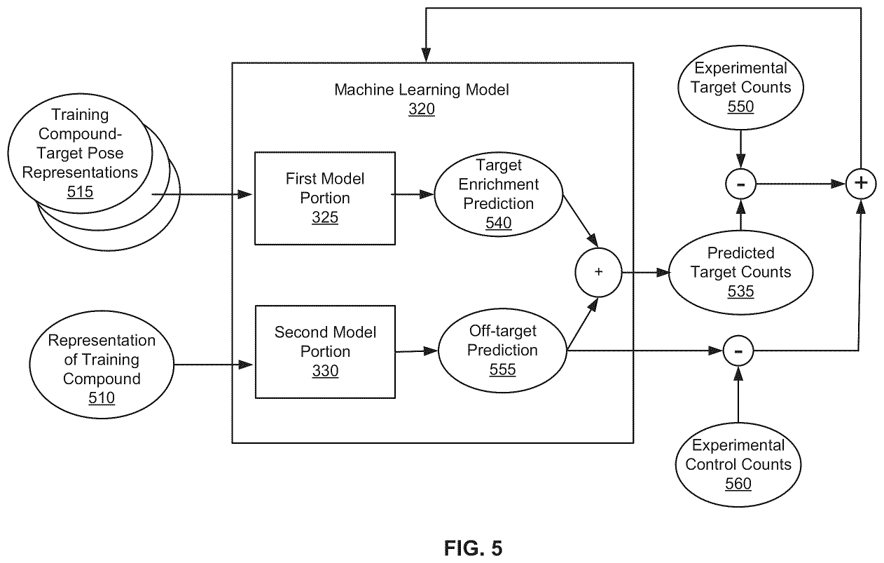

Reference is now made to FIG. 5, which depicts an example flow diagram for training the machine learning model, in accordance with an embodiment. Specifically, FIG. 5 depicts a single training iteration for a training compound. Thus, the flow diagram shown in FIG. 5 can be performed multiple times over multiple iterations to train the machine learning model.

The example flow diagram begins with a plurality of training compound-target pose representations 515 (also referred to herein as representations of training compound-target poses) and a representation of a training compound 510. The representation of the training compound 510 may be a transformation of a fingerprint (e.g., a Morgan fingerprint) of the training compound. For example, the representation of the training compound 510 may be a fingerprint embedding generated by applying a multilayer perceptron (MLP) to the fingerprint of the training compound 510.

In various embodiments, the plurality of training compound-target pose representations 515 are generated by performing an in silico molecular docking analysis to generate a plurality of training compound-target poses, followed by featurization of the plurality of training compound-target poses. In various embodiments, featurization of the plurality of training compound-target poses includes applying a neural network model (e.g., GNINA convolutional neural network) to identify the features. The features of the plurality of training compound-target poses are combined with the representation of the training compound 510 to generate the training compound-target pose representations 515.

As shown in FIG. 5, both the training compound-target pose representations 515 and the representation of the training compound 510 are provided as input to the machine learning model 320. In particular, the machine learning model 320 includes a first model portion 325 and a second model portion. Here, the first model portion 325 analyzes the training compound target pose representations 515 to generate a target enrichment prediction 540 representing binding between the training compound and a target (e.g., protein target). The second model portion 330 analyzes the representation of the training compound 510 to generate an off-target prediction (e.g., a noise prediction) 555.

The target enrichment prediction 540 and the off-target prediction 555 are combined to generate the predicted target counts 535. Here, the target enrichment prediction 540 represents a learned enrichment value representing binding between the training compound and the target, absent sources of noise (e.g., background, matrix, covariates). The off-target prediction 555 represents a learned value or score attributable to sources of non-target binding and/or other noise sources (e.g., background, matrix, covariates). The predicted target counts 535 represents a prediction of DEL counts of a DEL panning experiment in which various sources of non-target binding and/or other sources of noise (e.g., background, matrix, covariates) are included. In various embodiments, combining the target enrichment prediction 540 and the off-target prediction 555 involves summing the target enrichment prediction 540 and the off-target prediction 555. In various embodiments, combining the target enrichment prediction 540 and the off-target prediction 555 involves performing a linear or non-linear combination of the target enrichment prediction 540 and the off-target prediction 555. For example, in some embodiments, combining the target enrichment prediction 540 and the off-target prediction 555 may involve performing a weighted summation of the target enrichment prediction 540 and the off-target prediction 555, where the weights are previously learned (e.g., learned weights from a machine learning model, such as a neural work) or can be fixed weights determined according to a predetermined weighting scheme.

Given the predicted target counts 535 and the off-target prediction 555, a loss value is calculated. Here, the loss value can be calculated based on a combination of the predicted target counts 535, the experimental target counts 550, the off-target prediction 555, and the experimental control counts 560. For example, as shown in FIG. 5, the loss value can be calculated based on a combination of 1) a difference between the predicted target counts 535 and the experimental target counts 550 and 2) a difference between the off-target prediction 555 and the experimental control counts 560. In various embodiments, the loss value is calculated using a negative log likelihood loss function of zero-inflated Poisson (ZIP) distributions modeling the experimental target counts 550 and the experimental control counts 560.

The loss value is backpropagated to further train the machine learning model 320. Specifically, the parameters of the machine learning model 320 (e.g., parameters of the first model portion 325 and parameters of the second model portion 330) are adjusted according to the calculated loss value.

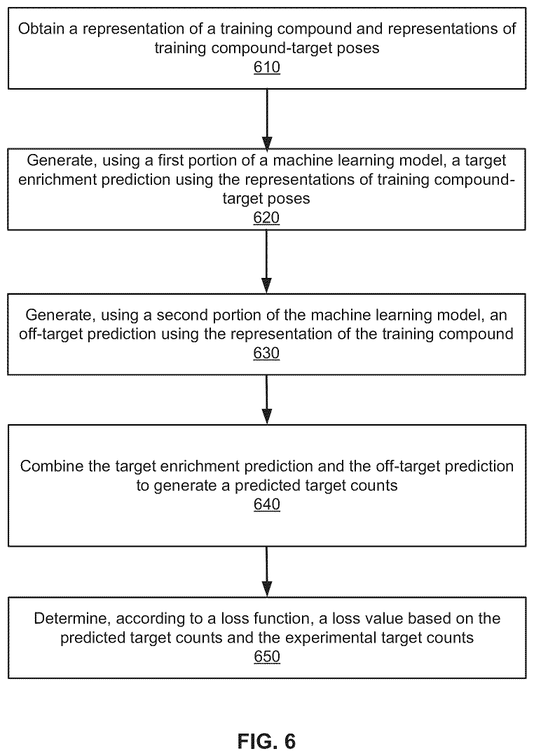

Reference is now made to FIG. 6, which depicts an example flow process for training a machine learning model, in accordance with an embodiment.

Step 610 involves obtaining a representation of a training compound and representations of training compound-target poses. In various embodiments, the representation of the training compound is a fingerprint or a transformation of a fingerprint, such as a Morgan fingerprint or a transformation of a Morgan fingerprint, of the training compound. The representations of training compound-target poses are generated by combining a representation of the training compound with features of a plurality of predicted training compound-target poses. In particular embodiments, features of a plurality of predicted training compound-target poses are generated by applying a pretrained model, such as a neural network model (e.g., GNINA convolutional neural network) to the plurality of the predicted compound-target poses, which represent possible 3D configurations of the compound when bound to the target.

Step 620 involves generating, using a first portion of a machine learning model, a target enrichment prediction using the representations of training compound-target poses. Step 630 involves generating, using a second portion of the machine learning model, an off-target prediction (e.g., from non-target binding and/or other sources of noise) using the representation of the training compound.

Step 640 involves combining the target enrichment prediction and the off-target prediction to generate a predicted target counts. Step 650 involves determining, according to a loss function, a loss value based on the predicted target counts and the experimental target counts. In various embodiments, the loss value is further determined based on the off-target prediction and the experimental control counts.

Generally, using the loss value, the parameters of the machine learning model can be tuned to improve the predictive capacity of the model. For example, over training iterations, the target enrichment prediction is learnt by trying to predict the experimental control counts (e.g., observed experimental control counts from a DEL experiment modeling a particular covariate) and the experimental target counts (e.g., observed experimental counts from a target DEL experiment, which further includes counts arising from background, matrix, and other covariates).

### Systems and Computing Devices

In various embodiments, the methods described herein, including the methods of training and deploying machine learning models are performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

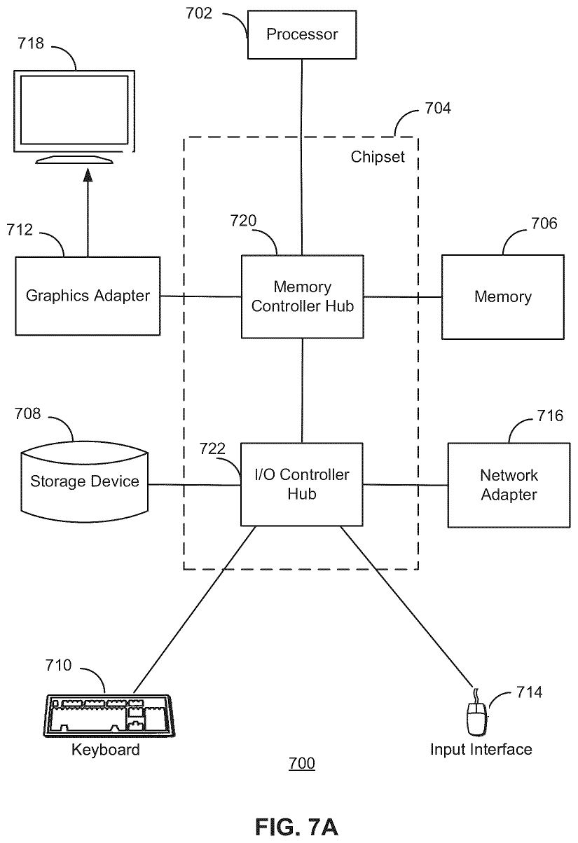

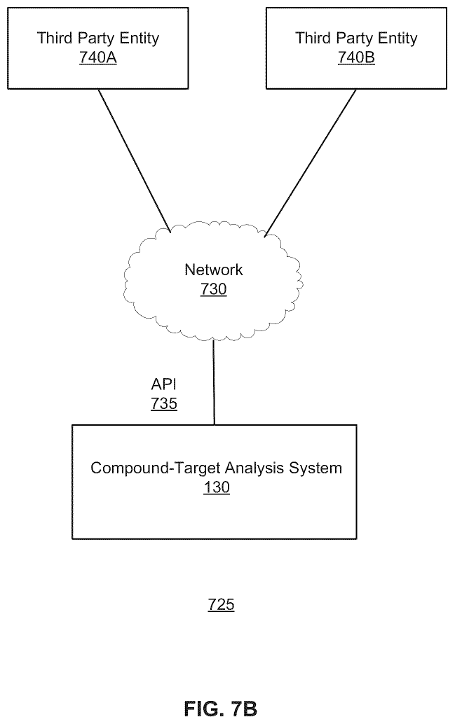

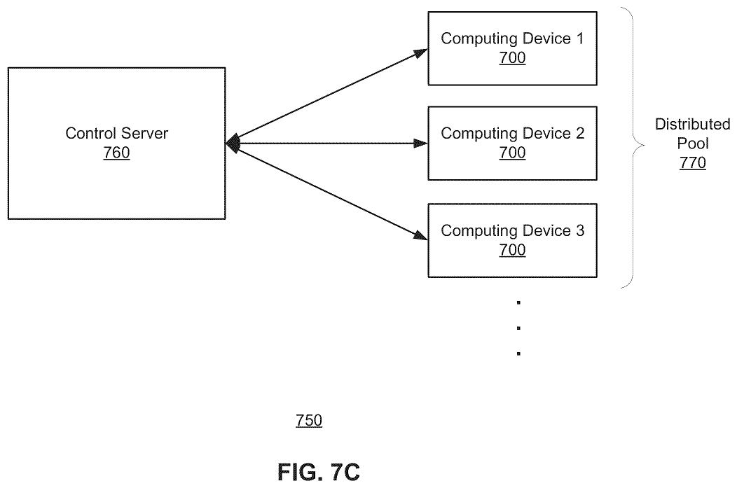

FIG. 7A illustrates an example computing device for implementing system and methods described in FIGS. 1A-1B, 2, 3A-3B, 4, 5, and 6. Furthermore, FIG. 7B depicts an overall system environment for implementing a compound-target analysis system, in accordance with an embodiment. FIG. 7C is an example depiction of a distributed computing system environment for implementing the system environment of FIG. 7B.

In some embodiments, the computing device 700 shown in FIG. 7A includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and network adapter 716 are coupled to the I/O controller hub 722. Other embodiments of the computing device 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of input interface, a keyboard, or some combination thereof, and is used to input data into the computing device 700. In some embodiments, the computing device 700 may be configured to receive input (e.g., commands) from the input interface 714 via gestures from the user. The graphics adapter 712 displays images and other information on the display 718. The network adapter 716 couples the computing device 700 to one or more computer networks.

The computing device 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computing devices 700 can vary from the embodiments described herein. For example, the computing device 700 can lack some of the components described above, such as graphics adapters 712, input interface 714, and displays 718. In some embodiments, a computing device 700 can include a processor 702 for executing instructions stored on a memory 706.

In various embodiments, the different entities depicted in FIG. 7B may implement one or more computing devices to perform the methods described above, including the methods of training and deploying one or more machine learning models. For example, the compound-target analysis system 130, third party entity 740A, and third party entity 740B may each employ one or more computing devices. As another example, one or more of the sub-systems of the compound-target analysis system 130 (as shown in FIG. 1B) may employ one or more computing devices to perform the methods described above.

The methods of training and deploying one or more machine learning models can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a machine learning model disclosed herein.

Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that is capable of recording and reproducing the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

### System Environment

FIG. 7B depicts an overall system environment for implementing a compound-target analysis system, in accordance with an embodiment. The overall system environment 725 includes a compound-target analysis system 130, as described earlier in reference to FIG. 1A, and one or more third party entities 740A and 740B in communication with one another through a network 730. FIG. 7A depicts one embodiment of the overall system environment 700. In other embodiments, additional or fewer third party entities 740 in communication with the compound-target analysis system 130 can be included. Generally, the compound-target analysis system 130 implements machine learning models that make predictions, e.g., predictions for compound binding, virtual screen, or hit selection and analysis. The third party entities 740 communicate with the compound-target analysis system 130 for purposes associated with implementing the machine learning models or obtaining predictions or results from the machine learning models.

In various embodiments, the methods described above as being performed by the compound-target analysis system 130 can be dispersed between the compound-target analysis system 130 and third party entities 740. For example, a third party entity 740A or 740B can generate training data and/or train a machine learning model. The compound-target analysis system 130 can then deploy the machine learning model to generate predictions e.g., predictions for compound binding, virtual screen, or hit selection and analysis.

### Third Party Entity

In various embodiments, the third party entity 740 represents a partner entity of the compound-target analysis system 130 that operates either upstream or downstream of the compound-target analysis system 130. As one example, the third party entity 740 operates upstream of the compound-target analysis system 130 and provide information to the compound-target analysis system 130 to enable the training of machine learning models. In this scenario, the compound-target analysis system 130 receives data, such as DEL experimental data collected by the third party entity 740. For example, the third party entity 740 may have performed the analysis concerning one or more DEL experiments (e.g., DEL experiment 115A or 115B shown in FIG. 1A) and provides the DEL experimental data of those experiments to the compound-target analysis system 130. Here, the third party entity 740 may synthesize the small molecule compounds of the DEL, incubate the small molecule compounds of the DEL with immobilized protein targets, eluting bound compounds, and amplifying/sequencing the DNA tags to identify putative binders. Thus, the third party entity 740 may provide the sequencing data to the compound-target analysis system 130.

As another example, the third party entity 740 operates downstream of the compound-target analysis system 130. In this scenario, the compound-target analysis system 130 may identify predicted binders through a virtual screen and provides information relating to the predicted binders to the third party entity 740. The third party entity 740 can subsequently use the information identifying the predicted binders relating for their own purposes. For example, the third party entity 740 may be a drug developer. Therefore, the drug developer can synthesize the predicted binder for further investigation.

### Network

This disclosure contemplates any suitable network 730 that enables connection between the compound-target analysis system 130 and third party entities 740. The network 730 may comprise any combination of local area and/or wide area networks, using both wired and/or wireless communication systems. In one embodiment, the network 730 uses standard communications technologies and/or protocols. For example, the network 730 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 730 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 730 may be represented using any suitable format, such as hypertext markup language (HTML) or extensible markup language (XML). In some embodiments, all or some of the communication links of the network 730 may be encrypted using any suitable technique or techniques.

### Application Programming Interface (API)

In various embodiments, the compound-target analysis system 130 communicates with third party entities 740A or 740B through one or more application programming interfaces (API) 735. The API 735 may define the data fields, calling protocols and functionality exchanges between computing systems maintained by third party entities 740 and the compound-target analysis system 130. The API 735 may be implemented to define or control the parameters for data to be received or provided by a third party entity 740 and data to be received or provided by the compound-target analysis system 130. For instance, the API may be implemented to provide access only to information generated by one of the subsystems comprising the compound-target analysis system 130. The API 735 may support implementation of licensing restrictions and tracking mechanisms for information provided by compound-target analysis system 130 to a third party entity 740. Such licensing restrictions and tracking mechanisms supported by API 735 may be implemented using blockchain-based networks, secure ledgers and information management keys. Examples of APIs include remote APIs, web APIs, operating system APIs, or software application APIs.

An API may be provided in the form of a library that includes specifications for routines, data structures, object classes, and variables. In other cases, an API may be provided as a specification of remote calls exposed to the API consumers. An API specification may take many forms, including an international standard such as POSIX, vendor documentation such as the Microsoft Windows API, or the libraries of a programming language, e.g., Standard Template Library in C++ or Java API. In various embodiments, the compound-target analysis system 130 includes a set of custom API that is developed specifically for the compound-target analysis system 130 or the subsystems of the compound-target analysis system 130.

### Distributed Computing Environment

In some embodiments, the methods described above, including the methods of training and implementing one or more machine learning models, are, performed in distributed computing system environments where local and remote computer systems, which are linked (either by hardwired data links, wireless data links, or by a combination of hardwired and wireless data links) through a network, both perform tasks. In some embodiments, one or more processors for implementing the methods described above may be located in a single geographic location (e.g., within a home environment, an office environment, or a server farm). In various embodiments, one or more processors for implementing the methods described above may be distributed across a number of geographic locations. In a distributed computing system environment, program modules may be located in both local and remote memory storage devices.

FIG. 7C is an example depiction of a distributed computing system environment for implementing the system environment of FIG. 7B. The distributed computing system environment 750 can include a control server 760 connected via communications network with at least one distributed pool 770 of computing resources, such as computing devices 700, examples of which are described above in reference to FIG. 7. In various embodiments, additional distributed pools 770 may exist in conjunction with the control server 760 within the distributed computing system environment 750. Computing resources can be dedicated for the exclusive use in the distributed pool 770 or shared with other pools within the distributed processing system and with other applications outside of the distributed processing system. Furthermore, the computing resources in distributed pool 770 can be allocated dynamically, with computing devices 700 added or removed from the pool 770 as necessary.

In various embodiments, the control server 760 is a software application that provides the control and monitoring of the computing devices 700 in the distributed pool 770. The control server 760 itself may be implemented on a computing device (e.g., computing device 700 described above in reference to FIG. 7A). Communications between the control server 760 and computing devices 700 in the distributed pool 770 can be facilitated through an application programming interface (API), such as a Web services API. In some embodiments, the control server 760 provides users with administration and computing resource management functions for controlling the distributed pool 770 (e.g., defining resource availability, submission, monitoring and control of tasks to performed by the computing devices 700, control timing of tasks to be completed, ranking task priorities, or storage/transmission of data resulting from completed tasks).

In various embodiments, the control server 760 identifies a computing task to be executed across the distributed computing system environment 750. The computing task can be divided into multiple work units that can be executed by the different computing devices 700 in the distributed pool 770. By dividing up and executing the computing task across the computing devices 700, the computing task can be effectively executed in parallel. This enables the completion of the task with increased performance (e.g., faster, less consumption of resources) in comparison to a non-distributed computing system environment.

In various embodiments, the computing devices 700 in the distributed pool 770 can be differently configured in order to ensure effective performance for their respective jobs. For example, a first set of computing devices 700 may be dedicated to performing collection and/or analysis of phenotypic assay data. A second set of computing devices 700 may be dedicated to performing the training of machine learning models. The first set of computing devices 700 may have less random access memory (RAM) and/or processors than the second set of second computing devices 700 given the likely need for more resources when training the machine learning models.

The computing devices 700 in the distributed pool 770 can perform, in parallel, each of their jobs and when completed, can store the results in a persistent storage and/or transmit the results back to the control server 760. The control server 760 can compile the results or, if needed, redistribute the results to the respective computing devices 700 to for continued processing.

In some embodiments, the distributed computing system environment 750 is implemented in a cloud computing environment. In this description, “cloud computing” is defined as a model for enabling on-demand network access to a shared set of configurable computing resources. For example, the control server 760 and the computing devices 700 of the distributed pool 770 may communicate through the cloud. Thus, in some embodiments, the control server 760 and computing devices 700 are located in geographically different locations. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### EXAMPLES

### Example 1: Example DEL Model (DEL-Dock)

The Examples describe the disclosed model, hereafter referred to as “DEL-Dock”, that directly learns a joint protein-ligand representation by synthesizing multi-modal information within a unified probabilistic framework to learn enrichment scores. This approach combines molecule-level descriptors with spatial information from docked protein-ligand complexes to explain DEL sequencing counts by delineating contributions from spurious matrix binding and target protein binding. By exposing the model to a collection of docked poses for each molecule, the model learned, without explicit supervision, to attribute importance to individual poses which serves as a bespoke scoring model for molecular docking pose selection. Separately DEL data and docked poses provided noisy signals of binding affinity, but these two data modalities were combined to construct a model that better learns the origins of binding within a molecular landscape.

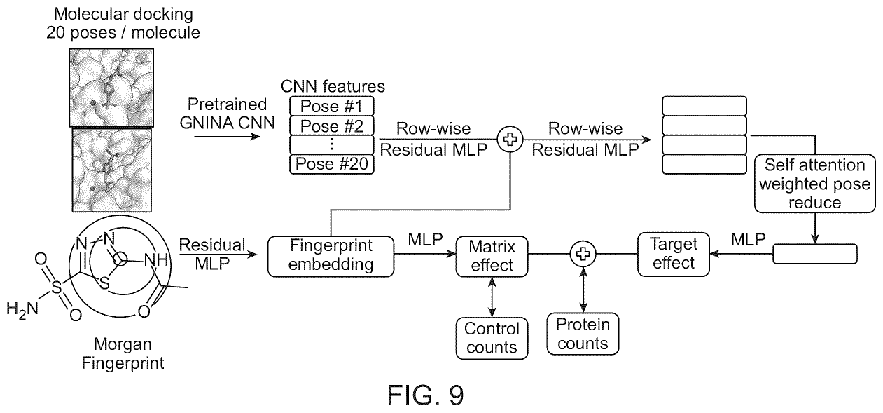

The model combines two different representational modalities, molecule-level descriptors and docked protein-ligand complexes, to capture the latent aspects of protein binding and spurious matrix binding through a probabilistic perspective. Specifically, FIG. 9 depicts a schematic illustration of the DEL-Dock neural network architecture and data flow.

The combinatorial construction of DELs motivated using expressive molecular representations to capture statistical correlation between the building block substructures used in DEL synthesis. Morgan fingerprints were calculated using RDKit version 2020.09.1,24 and serve as the basis for molecular representations. Molecular representations are standard descriptors for representational problems on small molecules and also provide the added benefit of their simple construction and rapid processing. Morgan fingerprints compute a structural bit hash of the molecule by enumerating k-hop substructures about each atom. Since there are many shared structural features across different molecular compounds, these fingerprints constituted a simple representation that has demonstrated empirical performance throughout cheminformatic domains. Docked protein-ligand poses were generated using a pretrained voxel-based CNN model from GNINA, which captures spatial relationships by discretizing space into three-dimensional voxels and leveraging CNNs to learn complex hierarchical representations. The class of CNN models used in this work was originally trained on the PDBBind database, capitalizing on this supervised data source to capture the features that characterize protein-ligand interactions.

Let X denote the set of molecules in the data, where each molecule x∈X has an associated set of n docked poses {p₁, p₂, . . . , pₙ}∈P and cᵢ^{m}∈C^{m}, cᵢ^{t}∈C^{t} are the ith replicate of count data from the beads-only control and target protein experiments respectively. Additionally, the following featurization transformations were defined that were used to construct the molecule and pose embeddings: Φ: X→[0, 1]ⁿ^{_{ϕ}} is the function that generates a n_{Φ}-bit molecular fingerprint; here, a 2048-bit Morgan fingerprint was implemented with radius 3. Ψ: X×P→Rⁿ^{_{Ψ}} is the transformation that outputs an embedding of the molecule and a specific spatial protein-ligand complex, where a pre-trained voxel-based CNN was used to perform this transformation.

Let h^{f}=MLP(Φ(x)) be the molecule embedding learned by the model, which is computed by applying a multilayer perceptron (MLP) to the fingerprint representation. Individual docked pose embeddings are similarly computed, with one difference being that the fingerprint embedding, hᵢ^{p}=MLP([Ψ(x, pᵢ); h^{f}]), is also incorporated into this representation.

To synthesize the set of poses for each molecule, a self-attention layer is applied over the pose embeddings. Attention weights are computed in accordance with Equation (1) where (w^{T}, W^{U}, W^{V}) are learnable weights, σ is the sigmoid activation and ⊙ is element-wise multiplication. The final output pose embedding that combines information from all input poses is then computed as a weighted embedding vector

$${h}^{p} = \frac{1}{n} {Σ}_{i} {a}_{i} {h}_{i}^{p} .$$

$${a}_{i} = \frac{exp [ {w}^{T} ( tanh ( {W}^{U} {h}_{i}^{p} ) ⊙ σ ( {W}^{V} {h}_{i}^{p} ) ) ]}{{\sum }_{j} exp [ {w}^{T} ( tanh ( {W}^{U} {h}_{j}^{p} ) ⊙ σ ( {W}^{V} {h}_{j}^{p} ) ) ]} \qquad ( Equation ( 1 ) )$$

Equipped with these molecule and pose embeddings, the model learns the contributions of both spurious matrix binding and target protein binding by predicting latent scores that strive to maximize the likelihood of the observed data under the model. Several distinct modeling choices are made to mirror assumptions about the data-generation process that accounts for various sources of experimental noise.

Matrix Binding is a confounding factor inherent to DEL experiments, since compounds are prone to binding to the multifarious components comprising the immobilized matrix in addition to the intended protein target. For each molecule x, a latent matrix binding score λₜ=f(h^{f}, h^{p}) is learned. Since matrix binding is not a function of the protein-ligand pose representation, the matrix binding enrichment remains only a function of the molecule embedding h^{f}.

Target Binding is learned through λₜ=f(h^{f}, h^{p}), jointly utilizing both molecule and pose representations. This design choice reflects that sequencing counts from the target protein experiment must be a function of both small molecule binding to the protein receptor, represented here as featurizations of the docked protein-ligand complexes, along with promiscuous binding to the immobilized matrix.

The observed count data for both the control and protein target experiments can be modeled as originating from underlying Poisson distributions, which naturally characterize any discrete count data from independently sampled events. Due to possible sequencing noise, this basic Poisson model is further augmented as a zero-inflated probability distribution. This design choice is motivated by the chance that sparse zero counts in the data could be explained as an artifact of imperfect sequencing technology. This assumption is directly incorporated into the structure of the model. Using zero-inflated distributions also allows for more flexibility in the experimental process—enabling models to explain zero-counts as an artifact of the data generation process, rather than an outcome of poor protein binding.

Let C be distributed as a zero-inflated Poisson (ZIP), its probability density function (PDF) is defined in (Eq. 2). Here, λ is the rate parameter of the underlying Poisson distribution, and is a function of the model's learned latent enrichments, while π denotes the occurrence of choosing the zero distribution, and is taken to be the empirical average. Here, since the matrix and target are modeled as separate count distributions, two distinct rate parameters are computed for each ZIP distribution as shown in Equation (3). Since the observed target counts are a function of both matrix binding and binding to the target, the rate parameter for the target distribution is a function of λₘ and λₜ. The final loss function is a typical negative log-likelihood (NLL) loss over the observed counts for both matrix and target counts Equation (4).

$$P ( C - c \vert λ , π ) = \{ \begin{matrix}π + ( 1 - π ) {e}^{- λ} & if c = 0 \\ ( 1 - π ) \frac{{λ}^{c} {e}^{- λ}}{c 1} & if c > 0\end{matrix} \qquad ( Equation ( 2 ) )$$

$${C}^{m} \sim ZIP ( {λ}_{m} , {π}_{m} ) {λ}_{m} = exp ( MLP ( {h}^{f} ) ) \qquad ( Equation ( 3 ) )$$

$${C}^{t} \sim ZIP ( {λ}_{t} , {π}_{t} ) {λ}_{t} = exp ( MLP ( {h}^{p} ) ) + {λ}_{m}$$

$$L = - {\sum }_{{c}_{i}} log [ P ( {C}^{m} = {c}_{i} \vert {λ}_{m} , {π}_{m} ) ] - {\sum }_{{c}_{j}} log [ P ( {C}^{t} = {c}_{j} \vert {λ}_{t} , {π}_{t} ) ] \qquad ( Equation ( 4 ) )$$

### Example 2: Example Training, Evaluation, and Implementation of DEL-Dock DEL and Evaluation Data

Training the model involved using publicly available DEL data that was collected as described in Gerry, C. J., et al. DNA barcoding a complete matrix of stereoisomeric small molecules. Journal of the American Chemical Society 2019, 141, 10225-10235, which is hereby incorporated by reference in its entirety. This tri-synthon library includes ˜100 k molecules with count data for panning experiments for the human carbonic anhydrase IX (CAIX) protein. In addition to on-target counts, the data include beads-only no-target controls. Four replicate sets of counts for the protein target experiments were provided, while two replicates of the control experiments were provided in this data set. To account for possible noise in different replicates, the counts were normalized for each target and control replicate by dividing each count by the sum of counts in that replicate experiment and then multiplying by 1×10⁶ to re-calibrate the scale of the counts. This data prepossessing provides the interpretation of each molecule count as a molecular frequency of that molecule within the DEL library. The processed data set is then used to train the models employing a 80/10/10—train/validation/test split.

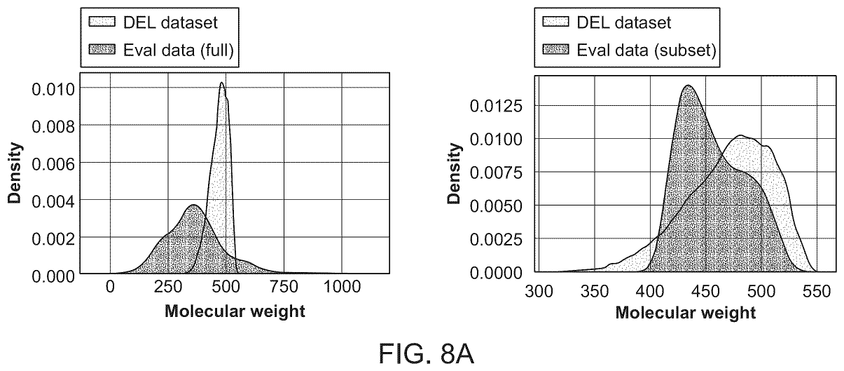

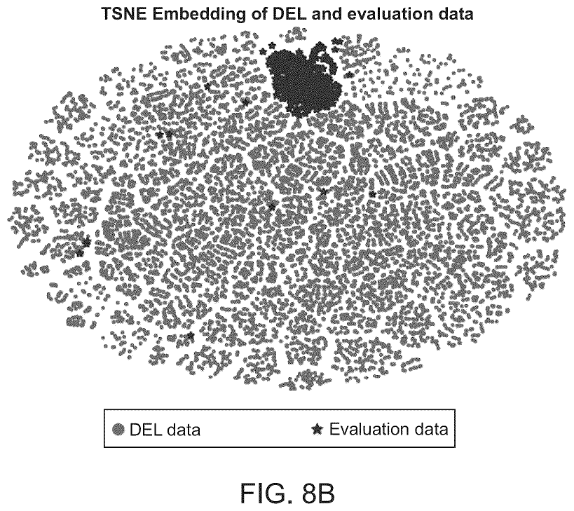

The performance of the models were evaluated on benchmarks using an external set of affinity measurements of small molecules curated from the BindingDB web database. Binding affinities were queried for the Human Carbonic anhydrase 9 (CAIX) protein target (UniProt: Q16790), and only molecules containing the same atom types as those present in the DEL data set (C, O, N, S, H, I) were kept. This external evaluation data set is composed of 3041 small molecules with molecular weights ranging from ˜25 atomic mass units (amu) to ˜1000 amu and associated experimental inhibitory constant (Ki) measurements ranging from ˜0.15 M to ˜90 pM. The median affinity value was used in the cases where multiple different affinity measurements were reported for the same molecule. Furthermore, a subset of this dataset was considered, which consists of the 521 molecules with molecular weights between 417 amu and 517 amu, a distribution of which is shown in the right panel of FIG. 8A. Specifically, FIG. 8A shows a comparison of the distribution of molecular weights between the DEL data set and the full evaluation data set (left) and the 417-517 amu subset of the evaluation data set (right). Distributions are generated as a Kernel Density Estimate (KDE) plot as implemented in seaborn. These molecular weights correspond to the interquartile range bounding the 10th and 90th percentiles of the molecular weights in the training dataset. This restricted subset presents a more challenging test as differentiation cannot rely on only extensive properties such as molecular weight, but must also effectively identify chemical motifs that impact molecular binding within this tightly bound range of molecular weights.

FIG. 8B shows a tSNE embedding of the DEL data set alongside the evaluation data. This tSNE embedding was generated by representing each molecule with a concatenation of three fingerprint representations: a 2048-dimensional Morgan fingerprint with a radius of 3, 167-dimensional MACCS (Molecular ACCess System) fingerprint, and finally a 2048-dimensional atom pair fingerprint. All fingerprints were calculated using RDKit. Sci-kit learn was then used to generate the tSNE embedding using a tanimoto similarity metric with a perplexity of 30 trained on the combined DEL and evaluation data. The evaluation data was largely isolated from the DEL data in this tSNE embedding, serving as an indication that the evaluation data is markedly different, or out of domain, than the DEL data used in training the models.

### Docking

Molecular docking was performed to generate a collection of ligand-bound poses to a target protein of interest for all molecules within the training and evaluation data sets. Docking was performed using the GNINA docking software employing the Vina scoring function. All molecules were docked against CAIX (PDB:5FL4) with the location of the binding pocket determined by the bound crystal structure ligand (9FK), using the default GNINA settings defining an 8×8×8 Å³ bounding box around this ligand. Initial three-dimensional conformers for all docked molecules were generated with RDKit. For each molecule, 20 docked poses were obtained from GNINA using an exhaustiveness parameter of 50, using the Vina scoring for end-to-end pose generation. This approach can similarly be performed using AutoDock Vina or Smina using the Vina scoring function.

### Training Settings

Featurizations for the docked poses were generated using pre-trained GNINA models provided in gnina-torch. The dense variant of the GNINA models composed of densely connected 3D residual CNN blocks were used to generate 224-dimensional embeddings of each docked pose. Morgan fingerprints for each molecule were calculated using RDKit with a radius of 3 embedded into a 2048 dimensional bit-vector.

All models were trained end-to-end using mini-batch gradient descent with the Adam optimizer and coefficients for the running averages of β₁=0.95 and β₂=0.999. A batch size of 64 was used with an initial learning rate of 1×10⁻⁴ and a linearly decaying learning rate scheduler where the learning rate is decayed by a factor of

$${γ}^{\frac{1}{{n}_{s t e p s}}}$$

every batch. For the learning rate scheduler, γ=0.1 and nₛₜₑₚₛ=1250, which corresponds to a 10× reduction in the learning rate after 1250 batches. Gradient clipping was additionally applied, where gradient norms were clipped to a maximum value of 0.1. During training, an exponential moving average was maintained over the model parameters which were updated each step with a decay rate of 0.999.

This exponential moving average version of the model parameters was then used for evaluation and throughout all inference tasks. LeakyReLU activation functions were used with a negative slope constant of 1×10⁻², except for the final activation function applied to the output logits corresponding to the matrix and target enrichment scores where an exponential function was applied as the terminal activation. A hidden dimensionality of 256 was used within MLP layers in the network. The residual MLP layers, which are responsible for processing the Morgan fingerprints along with the CNN features and embeddings (as shown in FIG. 9), are composed of 2 residually connected MLP layers using dropout with a probability of 0.5. The model in sum is composed of ˜1M parameters and is trained for 8 epochs on a single NVIDIA T4 GPU.

### Results

The model, which jointly combines topological features from the molecular graph and the spatial 3-D protein-ligand information, outperforms previous models on this task. Furthermore, the model is able to better rank ligand poses compared to traditional docking. The model learns latent binding affinity for each molecule to both the matrix and the target as the denoised signals compared to the observed count data. The higher enrichment scores predicted by the model are expected to be well-correlated with binding affinity, and therefore provide a useful metric for predicting anticipated protein binding in virtual screening campaigns.

A particular goal of this example, was to leverage the combinatorial scale of DEL data for transferable out-of-domain protein binding prediction. To test this capability, the model was first trained on DEL data screened against the human carbonic anhydrase IX (CAIX) protein target, and then used to predict enrichment scores for molecules with externally measured experimental binding affinities to CAIX. The performance of the model was evaluated in this setting by measuring spearman rank-correlation coefficients between predicted enrichments and the experimental affinity measurements (Table 1). Spearman rank-correlation is agnostic to the scale of the values. The model only restricts the enrichment scores to be positive quantities, with no specific distributional constraints, so spearman rank-correlation, which computes a correlation based only on the ordinal ranking of the predicted enrichments, is well suited for this test scenario.

Two partitions of the evaluation data set were considered: the full data set of 3041 molecules with associated inhibition constant (Kᵢ) measurements, and a 521 molecule subset of this data comprising of a restricted range molecular weights between approximately the 10th and 90th interquartile range of the DEL data set. Simple properties such as molecular weight or benzene sulfonamide presence, which is known to be an important binding motif for carbonic anhydrase, achieve better baseline performance on the full evaluation data compared to the restricted subset. These metrics suggest that this subset is more challenging as predictors must learn beyond these simple molecular properties to achieve good performance.

The trained model, which combines information from docked complexes with molecular descriptors outperformed previous techniques which only utilize one of these two data modalities. Traditional docking scores alone generated from AutoDock Vina result in the worst overall correlations, commensurate with previous observations that docking scores alone are typically not reliable predictors of binding affinity. Performance based on docked poses alone is however greatly improved when re-scoring the docked poses using pretrained GNINA CNN models. Another set of baselines are DEL models that rely only on molecular descriptors. For example, consider a simple model that involves training a random forest (RF) on the Morgan fingerprints using the enrichment metrics originally formulated to facilitate analysis of the DEL data set. The enrichment scores of molecules were predicted in the held-out dataset and the correlation with experimental Ki data. This baseline achieves reasonable performance on both the full and subset evaluation data, especially given the simplicity of the model. For a more sophisticated baseline, a Graph Neural Network (GNN) model was trained with a DEL-specific loss function. While this approach achieves good performance on the full evaluation data, correlations on the restricted subset are largely unchanged for all docking-based and molecular descriptor-based baselines.

The model disclosed herein, which combines docking pose embeddings with molecular fingerprint representations, outperforms all other baselines, with the largest improvements of ˜2× better spearman correlations than other approaches realized on the more challenging molecular weight restricted subset. Further ablation studies of the model are described herein.

TABLE 1
Comparison of spearman rank-correlation coefficients between predicted
affinity scores and experimental inhibition constant (Ki) measurements
curated from BindingDB. Spearman correlations are shown for the
complete 3041-molecule data set (full), and a 521-molecule subset
of this full data set confined to molecular weights between 417-
517 amu. This molecular weight range approximately corresponds to
the 10th and 90th interquartile range of the molecular weights spanned
by the DEL data set. Error bars are reported as standard deviations
over five independently initialized models.

| | Spearman Ki | Spearman Ki |
|---|---|---|
| Model | (full) ↓ | (subset) ↓ |
| Molecular weight | −0.121 | 0.074 |
| Benzenesulfonamide presence | −0.199 | −0.063 |
| Top Vina docking score Top | −0.068 | 0.119 |
| GNINA docking score | −0.279 ± 0.044 | −0.091 ± 0.061 |
| RF trained on enrichment | −0.231 ± 0.007 | −0.091 ± 0.012 |
| scores from (Gerry et al.) | | |
| GNN (Lim et al.) | −0.298 ± 0.005 | −0.075 ± 0.011 |
| DEL-Dock | −0.328 ± 0.01 | −0.186 ± 0.01 |

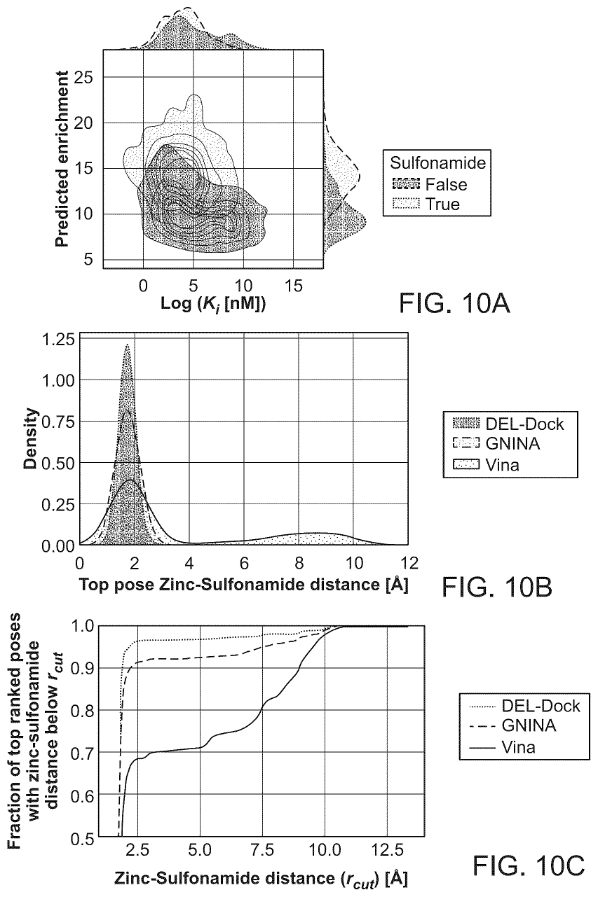

While the DEL-dock approach displays good prediction accuracy with respect to experimental binding measurements, the model provides further insights into the structural and chemical factors that influence binding. Compounds containing benzenesulfonamide have been well established in literature as the primarily chemical motif that drives small molecule binding to carbonic anhydrase. Though this was not explicitly incorporated as a learning signal for the model, the model was able to learn this association. Specifically, FIG. 10A is a visual depiction showing that the model predicts sulfonamides within the evaluation data set as more highly enriched compared to molecules which do not contain benzenesulfonamides. Interestingly, a comparatively large fraction of non-benzenesulfonamides were identified as good binders with low experimental Kᵢ. The elevated population of highly enriched non-benzenesulfonamides in this data set could be an artifact of publication bias in scientific literature. The model is ultimately trained on DEL data and therefore is expected to reflect underlying biases and idiosyncrasies of the data generation process. The most notable difference lies in that DEL experiments are only capable of measuring on-DNA binding, while the evaluation data are measurements of off-DNA binding. Nevertheless, the clear delineation of benzenesulfonamides in the predicted enrichments provides good post-hoc evidence that the model correctly identifies this important binding motif for this protein target.

An important structural component of benzenesulfonamides binding to carbonic anhydrase is coordination of the sulfonamide group with the zinc ion buried within the active site. In the vast majority of cases, one would then expect docking scoring functions to highly score poses that reflect this anticipated binding mode. As the model performs self-attention over pose embeddings, which are used to learn molecules' enrichment scores, the magnitude of the attention probabilities can be interpreted as the importance weight of that particular pose. FIG. 10B shows a distribution of zinc-sulfonamide distances for the top-selected docked pose comparing AutoDock Vina, GNINA, and the DEL-dock method (labeled as “DEL-Dock”) for all 1581 benzenesulfonamides-containing molecules in the evaluation data set. An alternate view of this data is presented in FIG. 10C, which shows the fraction of top-selected poses with zinc-sulfonamide distances below a distance threshold. This can effectively be interpreted as the cumulative distribution function (CDF) of the appropriately normalized associated probability distribution function (PDF) in FIG. 10B.

The AutoDock Vina scoring function exhibits the largest spread of zinc-sulfonamide distances, and as a result identifying a comparatively large fraction of poses as incorrectly coordinated. GNINA pose selection performs significantly better in this setting, identifying a larger fraction of well-coordinated poses with low zinc-sulfonamide distance. The DEL-dock method ultimately correctly coordinates the largest proportion of poses when compared to AutoDock Vina or GNINA. This approach for binding pose selection is markedly different than the approach taken by GNINA, which involves a separate pose scoring head trained to identify poses with low-RMSD to ground truth crystal structures. The attention scores are effectively latent variables trained only via the auxiliary task of modeling DEL data. The benefit of this approach is that good poses can be identified in an unsupervised manner, without requiring scarce and expensive crystal structures to serve as the source of supervision for pose selection.

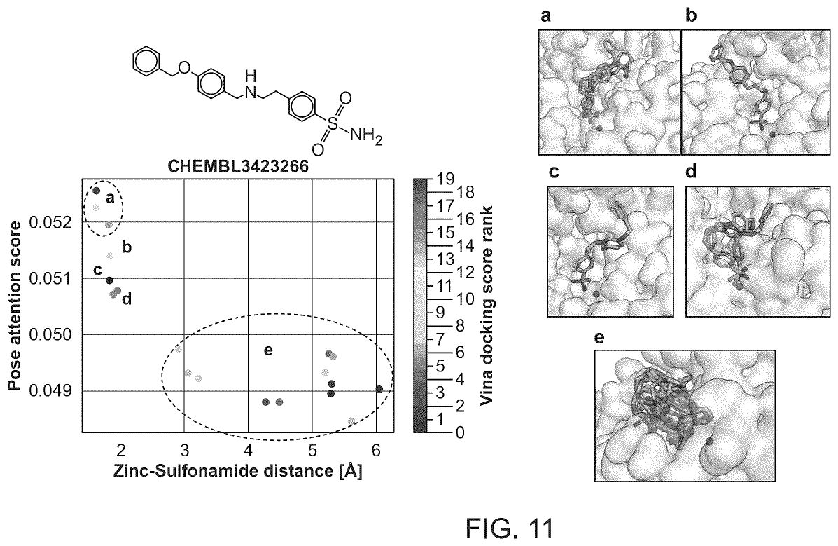

Lastly, the interpretability of the model was demonstrated by examining the distribution of attention scores learned by the model for a specific molecule. For this molecule, only 7 out of 20 docked poses correctly coordinate the sulfonamide group with the zinc ion buried in the protein active site. The model appropriately identifies this binding mode and learns attention scores that more favorably rank these 7 correctly coordinated poses. Specifically, FIG. 11 shows an analysis of pose attention scores for a representative molecule in the evaluation data set. The left panel of FIG. 11 shows that the model predicted pose attention scores plotted against the zinc sulfonamide distance of the docked pose and colored according to the ranking determined by the AutoDock Vina scoring function. The right panel of FIG. 11 shows different protein-ligand complexes which are visualized to show that the model highly ranks the conformers with zinc-sulfonamide coordination (A-D), while the conformers without the correct coordination are ranked lower (E).

The top-three ranked poses by the model have very similar conformations, each exhibiting zinc-sulfonamide coordination, and differing only in the orientation of the terminal benzene ring that is distant from the active site. The other poses that show zinc-sulfonamide coordination (e.g., the poses shown in the right panel of FIG. 11) are also ranked highly by the model, however, these poses exhibit less favorable conformations in several ways. For instance, in the right panel of FIG. 11, the conformation labeled as “b” is more exposed, and less protected by the protein. Finally, the model in general more poorly ranks poses that display incorrect zinc-sulfonamide coordination, as is shown in the conformation labeled in the right panel of FIG. 11 as “e”. These conformations typically have the terminal benzene ring inserted into the active site. Also, these poses reveal why zinc-sulfonamide distances alone can be a deceiving metric as some poses are capable of achieving low zinc-sulfonamide distances (˜3° A) due to the molecule “curling in” on itself within the active site. Nevertheless, the model recognizes this spurious binding mode and poorly ranks these poses with comparatively low attention scores, even though AutoDock Vina highly ranks many of these bad poses. Overall, the hierarchy of pose rankings by the model were commensurate with anticipated binding behavior for this protein target.

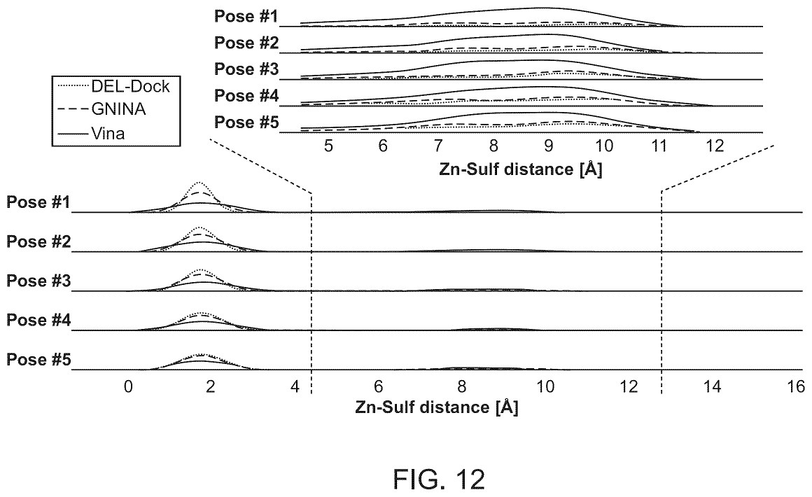

Reference is further made to FIG. 12, which shows the distributions of zinc-sulfonamide distances throughout the top five ranking poses as identified by the DEL-DOCK model attention scores, GNINA pose selection, and the AutoDock Vina scoring function. The highly ranked poses by the model attribute more density in the closely separated regime under ˜4° A than GNINA or Vina, and as a direct consequence of this fewer poses are selected by the model showing large separations between ˜4° A-˜13° A.

### Ablations

Disclosed in this section are a number of ablations to the model, which explore different architectural components and design choices (Table 2). First, training models with only fingerprint representations, without incorporating any information from the docked poses, resulted in a marked decrease in performance. On the other hand, models, trained using only CNN representations, performed much better and displayed comparable performance to only GNINA pre-trained models (Table 1). This represents an intuitive result, as this training setting is effectively equivalent to fine-tuning GNINA using a multi-instance learning over the pose representations. Interestingly, training on the CNN features alone already achieves good binding pose selection based on the latent attention scores, a feature the model was evidently capable of learning in isolation of the fingerprint representations. Also shown as a baseline is the MLP network trained using the bespoke loss function for modeling DEL data presented by Lim, K. S., et al. Machine learning on DNA-encoded library count data using an uncertainty-aware probabilistic loss function. Journal of Chemical Information and Modeling 2022.

Using a zero-inflated loss appears to impact performance, resulting in ˜25% increase in spearman rank-correlation on the full evaluation set and greater than a 2× increase on the subset. This performance jump could be related to the disparity in zero-counts between the control and on-target experiment: the control experiments have a zero-count frequency of ˜0.75% while the protein target experiments have a zero-count frequency of ˜55%. Using a zero-inflated distribution could provide the model more flexibility to explain zero-counts as an artifact of the data generation process, rather than an outcome of poor protein binding.

TABLE 2
Model ablations and other baselines. Error bars are calculated as
standard deviations over five independently initialized models.

| | Spearman Ki | Spearman Ki |
|---|---|---|
| Model | (full) ↓ | (subset) ↓ |
| Only fingerprints | −0.191 ± 0.005 | −0.083 ± 0.019 |
| Only CNN | −0.287 ± 0.005 | −0.124 ± 0.006 |
| MLP from Lim et al. | −0.244 ± 0.004 | −0.076 ± 0.017 |
| Without zero-inflated distribution | −0.26 ± 0.02 | −0.08 ± 0.03 |
| End-to-end voxels with frozen CNN | −0.278 ± 0.022 | −0.16 ± 0.03 |

Instead of using pre-computed CNN features from GNINA, the model was also trained directly from voxel representations using frozen CNN featurizers. The benefit of this approach is the ability to use data augmentation via random rotations and translations to implicitly enforce that the learned CNN embeddings remain roto-translationally equivariant. While the performance on the evaluation subset is comparable to the model trained on pre-computed CNN features (Table 1), the performance on the full data set is slightly reduced. This result could be due to the computational challenges of using voxelized representations. In particular, when training over many docked poses (in this case 20 poses per molecule), the batch size is effectively 20× larger—which presents a significant memory bottleneck as the voxel representation requires storing a 48×48×48×28 molecular grids (three dimensions discretizing space, and one for different atom types). Furthermore, the pre-computed features were already being generated with pre-trained CNN featurizers that have been trained using data augmentation, albeit on PDBBind for the separate task of affinity and pose prediction.

Lastly, presented in Table 3 is a comparison of spearman rank-correlation performances training on variable numbers of poses. For each model, the top-k poses generated via docking were used for training. Performance tends to generally improve with increasing number of poses used for training, with the largest difference in improvements realized on the molecular weight restricted subset. Beyond ˜10 poses appears to result in diminishing returns, in comparison to the jump in improvements seen from 2→10 poses.

TABLE 3
Model ablations training on different numbers of docked
poses. Error bars are calculated as standard deviations
over five independently initialized models.

| | | |
|---|---|---|
| Number of | Spearman Ki | Spearman Ki |
| training poses | (full) ↓ | (subset) ↓ |

| | | | |
|---|---|---|---|
| 2 | poses | −0.278 ± 0.011 | −0.112 ± 0.023 |
| 5 | poses | −0.304 ± 0.01 | −0.15 ± 0.02 |
| 10 | poses | −0.318 ± 0.007 | −0.175 ± 0.02 |
| 15 | poses | −0.324 ± 0.008 | −0.182 ± 0.014 |
| 20 | poses | −0.328 ± 0.009 | −0.186 ± 0.013 |

### CONCLUSIONS

This work presents an approach for modeling DEL data that combines docking-based and molecular descriptor-based data modalities. The DEL-dock approach involves predicting two interleaved quantities, enrichment scores, that explain the sequencing counts of the panning experiment measurements for both the on-target protein and the off-target control beads.

The model was first trained on DEL data screened against the human carbonic anhydrase (CAIX) protein target, and then implemented to predict binding for unseen molecules with external experimental constant of inhibition (Ki) affinity measurements curated from the BindingDB web database. For this prediction task, the DEL-Dock approach outperforms previous docking and DEL modeling techniques that only use either docked poses or molecular descriptor information alone. Furthermore, the model involves performing self-attention over pose embedding to learn over the set of possible poses. Analyzing these latent attention scores, the model effectively identifies good, docked poses. Compared to docking pose selection using either AutoDock Vina or GNINA, the model more reliably selects poses displaying the appropriate zinc sulfonamide coordination which is known to be the predominant binding mode for carbonic anhydrase. The model is interestingly capable of learning good pose selection in an unsupervised manner, training only on the voluminous DEL data rather than requiring crystal structures to serve as the source of supervision.

1-88. (canceled)

89. A method for performing molecular screening of one or more compounds for binding to a target, the method comprising:
obtaining a representation of a compound;
obtaining a plurality of predicted compound-target poses and determining features of the plurality of the predicted compound-target poses;
combining the representation of the compound and the features of the plurality of the predicted compound-target poses to generate a plurality of representations of compound-target poses; and
analyzing, using a machine learning model, at least the plurality of representations of the compound-target poses to generate a target enrichment prediction representing binding between the compound and the target, and at least the representation of the compound to generate an off-target prediction.

90. The method of claim 89, wherein the machine learning model comprises:
a first portion trained to predict the target enrichment prediction from representations of compound-target poses; and
a second portion trained to generate an off-target prediction from the representation of the compound.

91. The method of claim 90, wherein one or both of the first portion and the second portion of the machine learning model comprise a multilayer perceptron (MLP).

92. The method of claim 89, further comprising predicting a measure of binding between the compound and the target using the target enrichment prediction.

93. The method of claim 89, wherein analyzing, using the machine learning model, at least the plurality of representations of the compound-target poses comprises:
analyzing, using a first portion of the machine learning model, the plurality of representations of the compound-target poses to identify one or more candidate compound-target poses representing likely 3D configurations of the compound when bound to the target.

94. The method of claim 93, wherein the first portion of the machine learning model comprises a self-attention layer comprising one or more learnable attention weights for analyzing at least the plurality of representations of the compound-target poses.

95. The method of claim 93, wherein the first portion of the machine learning model comprises a layer that pays equal attention to each of the plurality of representations of the compound-target poses.

96. The method of claim 89, wherein the off-target prediction arises from one or more covariates comprising any of non-specific binding via controls, off-target data, and noise.

97. The method of claim 96, wherein off-targets data comprise one or more of binding to beads, binding to streptavidin of the beads, binding to biotin, binding to gels, binding to DEL container surfaces.

98. The method of claim 96, wherein the noise comprise one or more of starting tag imbalance, experimental conditions, chemical reaction yields, side and truncated products, errors from the library synthesis, DNA affinity to target, sequencing depth, and sequencing noise.

99. The method of claim 90, wherein the first portion of the machine learning model and the second portion of the machine learning model are trained using one or more training compounds with corresponding DNA-encoded library (DEL) outputs.

100. The method of claim 99, wherein the corresponding DNA-encoded library (DEL) outputs for a training compound comprises:
control counts arising from a covariate determined through a first panning experiment; and
target counts determined through a second panning experiment.

101. The method of claim 100, wherein for one of the training compounds, the first portion of the machine learning model and the second portion of the machine learning model are trained by:
generating, by the first portion, a target enrichment prediction from representations of training compound-target poses, the representations of training compound-target poses generated by combining a representation of the training compound and features of a plurality of predicted training compound-target poses;
generating, by the second portion, an off-target prediction from a representation of the training compound;
combining the target enrichment prediction and the off-target prediction to generate a predicted target counts; and
determining, according to a loss function, a loss value based on the predicted target counts and the experimental target counts.

102. The method of claim 101, wherein the loss value is further determined based on the off-target predictions and the experimental control counts.

103. The method of claim 101, wherein the loss value is determined according to probability density functions that model the experimental target counts and the experimental control counts.

104. The method of claim 103, wherein the probability density functions are represented by any one of a Poisson distribution, Binomial distribution, Gamma distribution, Binomial-Poisson distribution, Gamma-Poisson distribution, or negative binomial distribution.

105. The method of claim 104, wherein the Poisson distribution is a zero-inflated Poisson distribution.

106. The method of claim 89, wherein the plurality of predicted compound-target poses comprises at least 20 compound-target poses.

107. The method of claim 89, further comprising:
identifying a common binding motif across a subset of the one or more compounds, wherein the compounds in the subset have predicted measures of binding above a threshold binding value.

108. A non-transitory computer readable medium comprising instructions that, when executed by a processor, cause the processor to:
obtain a representation of a compound;
obtain a plurality of predicted compound-target poses and determining features of the plurality of the predicted compound-target poses;
combine the representation of the compound and the features of the plurality of the predicted compound-target poses to generate a plurality of representations of compound-target poses; and
analyze, using a machine learning model, at least the plurality of representations of the compound-target poses to generate a target enrichment prediction representing binding between the compound and the target, and at least the representation of the compound to generate an off-target prediction.
